(19) United States
(12) Patent Application Publication
(10) Pub. No.: US 2020/0345633 A1
(43) Pub. Date: Nov. 5, 2020

(54) Pharmaceutical Compositions for Controlling and/or Reducing the Progression of Myopia

(71) Applicants: Minas Theodore Coroneo, Randwick, New South Wales (AU); Brien Holden Vision Institute Limited, Sydney, New South Wales (AU)

(72) Inventors: Minas Theodore Coroneo, Randwick, New South Wales (AU); Monica Jong, Kensington, New South Wales (AU); Padmaja Rajagopal Sankaridurg, Maroubra, New South Wales (AU); Earl Leo Smith, III, Houston, TX (US); Amandeep Kaur, Kingsford, New South Wales (AU)

(21) Appl. No.: 16/760,663

(22) PCT Filed: Nov. 2, 2018
(86) PCT No.: PCT/AU2018/051187

§ 371 Date: Apr. 30, 2020

Related U.S. Application Data
(60) Provisional application No. 62/581,112, filed on Nov. 3, 2017.

(51) Int. Cl. A61K 9/00; A61K 31/46; A61K 31/522

(52) U.S. Cl. CPC A61K 9/0051; A61K 31/522; A61K 31/46

(57) ABSTRACT

A pharmaceutical composition comprising a muscarinic antagonist and an adenosine antagonist for topical or ophthalmic application, and ophthalmic devices containing or delivering the same, and methods of using the same, for controlling and/or reducing the progression of myopia.

### CROSS REFERENCE TO RELATED APPLICATION

This application claims the benefit of priority from U.S. Provisional Application No. 62/581,112, filed Nov. 3, 2017. The foregoing related application, in its entirety, is incorporated herein by reference.

### TECHNICAL FIELD

This relates to a pharmaceutical composition of a muscarinic antagonist and a non-selective adenosine antagonist for topical or ocular application, and ophthalmic devices containing or delivering the same, and methods of using the same, for controlling and/or reducing the progression of myopia.

### BACKGROUND

Myopia (sometimes referred to as near-sightedness or short-sightedness) is a condition where there is a mismatch between the length of the eye and the optics of the eye resulting in the image being formed in front of the retina of the eye. This refractive type error results in blurred vision for distant objects, while close or near objects appear normal. Most frequently, the reason for the mismatch is that the eyeball length (sometimes referred to as axial length) is longer than the optics of the eye. The longer eyeball length is generally the result of excessive axial (or longitudinal) growth of the eye. The condition of myopia is commonly seen worldwide, although not uniformly. For example, while the prevalence of myopia in the United States and Europe is about 30-40% of the population, it has reached epidemic proportions in many other societies, particularly in east Asia where over 90% of teenagers and young adults are near sighted (Dolgin E., “The myopia boom. Short-sightedness is reaching epidemic proportions. Some scientists think they have found a reason why,” Nature (2015) 519:276-278). Comparatively, about 50 to 60 years ago, the prevalence in east Asia was about 10-30%. In addition, over the same period, it has been observed that the prevalence of high myopia (myopia worse than −5.00 diopters (D)) has increased from a few percent to approximately 20% in many East Asian countries (Morgan I G, He M., “An Important Step Forward in Myopia Prevention: Low-Dose Atropine,” Ophthalmology (2016) 123:232-3). Apart from inconvenience and expense involved in correcting for the blurred distance vision, there are consequences for the long-term health of myopic eyes in older individuals with an increased prevalence of developing further vision impairments, including myopic maculopathy, retinal detachment, glaucoma, and cataracts (Curtin B J., The Myopias: Basic Science and Clinical Management. Harper & Row, Philadelphia, Pa., 1985). Thus, there is a need to prevent the eye from progressing to higher levels of myopia. Over the years, a number of early preventative measures and interventions, ranging from the use of pharmaceutical, optical, and environmental interventions, were proposed and assessed to slow the progression of myopia. Of these, pharmaceutical interventions were generally more effective in slowing myopia.

With respect to pharmaceutical interventions, one compound that was observed to slow the progression of myopia was Atropine, a muscarinic antagonist (more specifically, a nonselective muscarinic acetylcholinergic antagonist) (Chua et al. Atropine for the treatment of childhood myopia, Ophthalmol, 2285-2291, 2006). Initially, atropine was used in concentrations of approximately 1% to slow myopia. However, concern related to the use of atropine was that the dose concentrations that were observed to be effective for slowing myopia also induced side effects. Use of Atropine in concentrations of 1% and 0.5% resulted in significant short term adverse effects of an enlarged cyclopleged pupil, photophobia (discomfort or sensitivity to light), glare, and an inability to read or see at near, and also considered to result in long term adverse effects, such as damage to the ocular structures (e.g., crystalline lens and retina due to increased light). In addition, certain side effects, such as allergies, were also reported. More significantly, on stopping atropine dosing, rebound of myopia occurred. Furthermore, concurrent with the use of atropine, the individual required to be prescribed with bifocal spectacles so that they can view clearly at distance and near. Due to the concerns related to the side effects associated with the use of elevated concentrations of atropine, lower concentrations of atropine were trialed (Chia et al., “Atropine for the treatment of childhood myopia: safety and efficacy of 0.5%, 0.1% and 0.01% doses,” Ophthalmology (2012) 119(2), 347-354; Chia A, Lu Q S, Tan D., “Five-year clinical trial on atropine for the treatment of myopia 2: myopia control with atropine 0.01% eyedrops,” Ophthalmology (2016) 123:391-9). From these trials, low-concentration topical atropine (0.01 wt. %) was considered to not induce side effects that were observed with higher concentrations of 1 wt. %, 0.5 wt. %, and even 0.1 wt. % atropine. With 0.01 wt. % atropine, a marked reduction in the rebound effect was also observed during washout after higher doses and resulted in fewer side effects as the pupil size increase was minimal and effect on accommodative amplitude not significant. However on closer examination, low dose atropine (0.01 wt %) although effective in reducing adverse effects was not effective in reducing axial length elongation (Yam et al. Low Concentration Atropine for Myopia Progression (LAMP) study: A randomized, double-blinded, placebo-controlled trial of 0.05 wt. %, 0.025 wt. % and 0.01 wt. % Atropine eye drops in myopia control, Ophthalmology, Epub ahead of print, 2018; Chia et al. Atropine for the treatment of childhood myopia: safety and efficacy of 0.5 wt. %, 0.1 wt. %, and 0.01 wt. % doses (Atropine for the Treatment of Myopia 2). Ophthalmology. 2012; 119(2):347-54). Thus there is a need for an therapeutically effective dose that reduces axial elongation of the eye without significant adverse/side effects.

Another pharmaceutical intervention observed to have some efficacy in slowing the progression of myopia is the compound 7-methylxanthine (sometimes referred to as 7-MX; a metabolite of caffeine and theobromine), which is an adenosine receptor antagonist (more specifically, a non-selective adenosine antagonist). This compound was described in U.S. Pat. No. 6,710,051, which is herein incorporated by reference in its entirety. The systemic dosage of 7-methylxanthine by oral administration in young myopic children for one year resulted in a slowing of the progression of myopia and retards axial eye growth without significant side effects (Trier K, Munk Ribel-Madsen S, Cui D, Bregger Christensen S., “Systemic 7-methylxanthine in retarding axial eye growth and myopia progression: a 36-month pilot study,” J Ocul Biol Dis Infor. (2008) 1:85-93; Nie H H, Huo I J, Yang X, Gao Z Y, Zeng J W, Trier K, Cui D M., “Effects of 7-methylxanthine on form-deprivation myopia in pigmented rabbits,” Int. J. Ophthalmol. (2012) 5:133-7; Cui D, Trier K, Zeng J, Wu K, Yu M, Hu J, Chen X, Ge J., “Effects of 7-methylxanthine on the sclera in form deprivation myopia in guinea pigs,” Acta Ophthalmol. (2011) 89:328-34; Trier K, Olsen E B, Kobayashi T, Ribel-Madsen SM, “Biochemical and ultrastructural changes in rabbit sclera after treatment with 7-methylxanthine, theobromine, acetazolamide, or L-ornithine,” Br. J. Ophthalmol. (1999) 83:1370-5). It was thought that 7-methylxanthine exerted its effect by action on the posterior sclera. However, aside from the metabolic issues associated with oral dosing that limit achieving maximal efficacy for eye treatment, oral use of medication for eye treatment also suffers from compliance issues.

Of note, although the action of muscarinic receptor antagonists were observed to have been enhanced with low doses of caffeine to inhibit haloperidol-induced catalepsy (Moo-Puc R E, Góngora-Alfaro J L, Alvarez-Cervera F J, Pineda J C, Arankowsky-Sandoval G, Heredia-López F., “Caffeine and muscarinic antagonists act in synergy to inhibit haloperidol-induced catalepsy,” Neuropharmacology (2003) 45:493-503), it is not known whether using caffeine (or related agents or other adenosine antagonists) as adjunctive ophthalmic therapy is capable of reducing the dose of muscarinic receptor antagonists required to slow the progression of myopia, and/or whether it is capable of mitigating the adverse side effects associated with muscarinic antagonist ophthalmic monotherapy. Caffeine is a non-selective adenosine receptor antagonist, and it was observed that systemic intake of caffeine enhances accommodation (Osei et al. Caffeine intake is associated with pupil dilation and enhanced accommodation. Eye, 31(4), 615-619, 2017). Caffeine has safely been used topically on the human eye in a concentration of 1 wt. % (Chandra P, Gaur A, Varma S., “Effect of caffeine on the intraocular pressure in patients with primary open angle glaucoma,” Clin Ophthalmol. (2011) 5:1623-9). Interestingly, 7-methylxanthine (sometimes referred to as 7-MX), a metabolite of caffeine has been used systemically, by oral administration, to treat myopia in animal models and in a human trial (Nie H H, Huo I J, Yang X, Gao Z Y, Zeng J W, Trier K, Cui D M., “Effects of 7-methylxanthine on form-deprivation myopia in pigmented rabbits,” Int. J. Ophthalmol. (2012) 5:133-7; Cui D, Trier K, Zeng J, Wu K, Yu M, Hu J, Chen X, Ge J., “Effects of 7-methylxanthine on the sclera in form deprivation myopia in guinea pigs,” Acta Ophthalmol. (2011) 89:328-34; and Trier K, Olsen E B, Kobayashi T, Ribel-Madsen S M., “Biochemical and ultrastructural changes in rabbit sclera after treatment with 7-methylxanthine, theobromine, acetazolamide, or L-ornithine,” Br J Ophthalmol. (1999) 83:1370-5). In addition, 7-methylxanthine, considered an adenosine antagonist with potential effects on neurotransmitter release (including GABA), has been observed to retard myopia progression and axial eye growth without significant side effects (Trier K, Munk Ribel-Madsen S, Cui D, Brogger Christensen S., “Systemic 7-methylxanthine in retarding axial eye growth and myopia progression: a 36-month pilot study,” J Ocul Biol Dis Infor. (2008) 1:85-93).

Accordingly, there is a need for a pharmaceutical composition, and ophthalmic devices containing or delivering the same, and methods of using the same, for effectively controlling and/or reducing the progression of myopia that also avoid or minimize adverse side effects relating to pupillary size or accommodation.

### Definitions

Terms are used herein as generally used in the art, unless otherwise defined in the following:

The term “myopic eye” is understood to refer to an eye that is already myopic, is pre myopic, or has a refractive condition that is progressing towards myopia.

The term “ophthalmic device” is understood to refer to an object that is placed on or resides in the eye. The device may provide optical correction. An ophthalmic device includes, but is not limited to, a contact lens(es), an ocular insert(s), a corneal onlay(s), a corneal inlay(s), a nano wafer(s), a liposome(s), a nanoparticle(s), a punctal plug(s), or a hydrogel matrix(ces) with microfluid reservoir.

The terms “treating” (or “treat” or “treatment”), unless otherwise specified, includes the generally accepted meaning which encompasses preventing, controlling, slowing, reducing, retarding, and/or mitigating, a symptom associated with a disease (e.g., myopia), progression of a disease (e.g., myopia, such as the progression of myopia in an eye of a patient), and/or a disease (e.g., myopia). Treatment may include therapeutic and/or prophylactic administration (e.g., of a pharmaceutical composition or an ophthalmic device, as disclosed herein). For example, treatment of an eye that is already myopic (or at risk of developing myopia), in a patient diagnosed as having myopia (high, moderate, or low) or pre-myopic (at risk at developing myopia), may include, but is not limited to, preventing, controlling, slowing, reducing, retarding, or mitigating, the progression of myopia, increasing choroidal thickness of an eye (e.g., a myopic eye, a pre-myopic eye, or an eye at risk of developing myopia), and/or reducing axial (or longitudinal) growth of an eye (e.g., a myopic eye, a pre-myopic eye, or an eye at risk of developing myopia) of a patient diagnosed as having myopia or at risk of developing myopia.

The term “muscarinic antagonist” or “muscarinic receptor antagonist” refers to agents that act on or block the muscarinic receptors to prevent or antagonize the action of cholinergic agents or muscarinic agonists or muscarinic receptor agonists.

The term “adenosine antagonist” or “adenosine receptor antagonist” refers to agents that act on or block the adenosine receptors to the prevent or antagonize action of adenosine agonists or adenosine receptor agonists.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

In certain embodiments, the subject is a mammal. In certain embodiments, the subject is a human. In certain embodiments, the subject is an adult human. In certain embodiments, the subject is a human child.

### SUMMARY

Some embodiments described herein may provide pharmaceutical compositions, ophthalmic devices, and methods of treatment, to prevent, control, slow, reduce, retard, and/or mitigate the progression of myopia.

In one aspect, provided herein is a pharmaceutical composition, comprising a muscarinic antagonist, for example, a low concentration of a muscarinic antagonist, and an adenosine antagonist.

In another aspect, provided herein is an ophthalmic device containing a pharmaceutical composition comprising a muscarinic antagonist, for example, a low concentration of a muscarinic antagonist, and an adenosine antagonist, wherein the ophthalmic device delivers the pharmaceutical composition in a sustained release manner.

In another aspect, provided herein is a method of treating myopia in a patient in need thereof, comprising administering a pharmaceutical composition comprising a muscarinic antagonist, for example, a low concentration of a muscarinic antagonist, and an adenosine antagonist.

In another aspect, provided herein is a method of treating myopia to prevent, slow, retard, control and/or mitigate the progression of myopia in an eye of a patient in need thereof, comprising administering a pharmaceutical composition comprising a muscarinic antagonist, for example, a low concentration of a muscarinic antagonist, and an adenosine antagonist.

In another aspect, provided herein is a method of treating myopia in a patient in need thereof, comprises administering a pharmaceutical composition comprising a muscarinic antagonist, for example, a low concentration of a muscarinic antagonist, and administering a pharmaceutical composition comprising an adenosine antagonist.

In another aspect, provided herein is a method to prevent, slow, retard, control and/or mitigate the progression of myopia in an eye of patient in need thereof, comprises administering a pharmaceutical composition comprising a muscarinic antagonist, for example, a low concentration of a muscarinic antagonist, and administering a pharmaceutical composition comprising an adenosine antagonist.

In another aspect, provided herein is a method of treating myopia in a patient in need thereof, comprises administering an ophthalmic device containing a pharmaceutical composition comprising a muscarinic antagonist, for example, a low concentration of a muscarinic antagonist, and an adenosine antagonist.

In another aspect, provided herein is a method to prevent, slow, retard, control and/or mitigate the progression of myopia in an eye of patient in need thereof, comprises administering an ophthalmic device containing a pharmaceutical composition comprising a muscarinic antagonist, for example, a low concentration of a muscarinic antagonist, and an adenosine antagonist.

In certain embodiments of the pharmaceutical composition, the ophthalmic device, or the method of treating, disclosed herein, the muscarinic antagonist is a nonselective muscarinic acetylcholinergic antagonist.

In certain embodiments of the pharmaceutical composition, the ophthalmic device, or the method of treating, disclosed herein, the muscarinic antagonist is an M1 selective antagonist.

In certain embodiments of the pharmaceutical composition, the ophthalmic device, or the method of treating, disclosed herein, the muscarinic antagonist is atropine, or a pharmaceutically acceptable salt thereof.

In certain embodiments of the pharmaceutical composition, the ophthalmic device, or the method of treating, disclosed herein, the muscarinic antagonist is tropine, or a pharmaceutically acceptable salt thereof.

In certain embodiments of the pharmaceutical composition, the ophthalmic device, or the method of treating, disclosed herein, the muscarinic antagonist is tropic acid.

In certain embodiments of the pharmaceutical composition, the ophthalmic device, or the method of treating, disclosed herein, the muscarinic antagonist is used in low concentrations. In certain embodiments, the muscarinic antagonist is used in low concentrations, for example, the muscarinic antagonist is atropine and is used in concentrations of less than 0.05 wt. %, relative to the pharmaceutical composition. In certain embodiments, the muscarinic antagonist is atropine used in concentrations of between less than about 0.05 wt. % to no less than 0.001 wt. %, relative to the pharmaceutical composition. In certain embodiments, the muscarinic antagonist is atropine used in concentrations of approximately 0.045 wt. % or less, relative to the pharmaceutical composition. In certain embodiments, the muscarinic antagonist is atropine and is used in concentrations of approximately 0.04 wt. % or less, relative to the pharmaceutical composition. In certain embodiments, the muscarinic antagonist is atropine and is used in concentrations of approximately 0.035 wt. % or less, relative to the pharmaceutical composition. In certain embodiments, the muscarinic antagonist is atropine and is used in concentrations of approximately 0.03 wt. % or less, relative to the pharmaceutical composition. In certain embodiments, the concentration of atropine is in the range of between less than 0.05 wt. % to 0.001 wt. %, such as, between approximately 0.045 wt. % to 0.001 wt. %, between approximately 0.04 wt. % to 0.001 wt. %, between approximately 0.035 wt. % to 0.001 wt. %, between approximately 0.03 wt. % to 0.001 wt. %, between approximately 0.025 wt. % to 0.001 wt. %, between approximately 0.02 wt. % to 0.001 wt. %, between approximately 0.015 wt. % to 0.001 wt. %, between approximately 0.01 wt. % to 0.001 wt. %, between <0.01 wt. % to 0.001 wt. %, between approximately 0.045 wt. % to 0.01 wt. %, between approximately 0.04 wt. % to 0.02 wt. %, between approximately 0.03 wt. % to 0.02 wt. %, or between approximately 0.03 wt. % to 0.01 wt. %, relative to the pharmaceutical composition.

In certain embodiments of the pharmaceutical composition, the ophthalmic device, or the method of treating, disclosed herein, the muscarinic antagonist is present in an amount in the range from between approximately 0.001 to less than 0.05 wt. %, such as, between approximately 0.001-0.045 wt. %, between approximately 0.001-0.04 wt. %, between approximately 0.001-0.035 wt. %, between approximately 0.001-0.03 wt. %, between approximately 0.001-0.025 wt. %, between approximately 0.001-0.02 wt. %, between approximately 0.001-0.015 wt %, between approximately 0.001-0.01 wt. %, between approximately 0.001-0.005 wt. %, between approximately 0.005-0.03 wt. %, between approximately 0.005-0.04 wt %, between approximately 0.01-0.03 wt %, between approximately 0.01-0.045 wt. %, between approximately 0.01-0.04 wt %, between approximately 0.02-0.04 wt. %, between approximately 0.02-0.03 wt. %, between approximately 0.015-0.025 wt. %, between approximately 0.015-0.03 wt. %, or between approximately 0.015-0.035 wt. %, relative to the pharmaceutical composition.

In certain embodiments of the pharmaceutical composition, the ophthalmic device, or the method of treating, disclosed herein, the adenosine antagonist is a non-selective adenosine antagonist.

In certain embodiments of the pharmaceutical composition, the ophthalmic device, or the method of treating, disclosed herein, the non-selective adenosine antagonist is a xanthine derivative, or a pharmaceutically acceptable salt thereof.

In certain embodiments of the pharmaceutical composition, the ophthalmic device, or the method of treating, disclosed herein, the non-selective adenosine antagonist is caffeine, or a pharmaceutically acceptable salt thereof.

In certain embodiments of the pharmaceutical composition, the ophthalmic device, or the method of treating, disclosed herein, the non-selective adenosine antagonist is caffeine citrate.

In certain embodiments of the pharmaceutical composition, the ophthalmic device, or the method of treating, disclosed herein, the non-selective adenosine antagonist is 7-methylxanthine, or a pharmaceutically acceptable salt thereof.

In certain embodiments of the pharmaceutical composition, the ophthalmic device, or the method of treating, disclosed herein, the adenosine antagonist is present in an amount in the range of between approximately 0.1-5.0 wt. %, between approximately 0.1-4.0 wt. %, between approximately 0.1-3.0 wt. %, between approximately 0.1-2.0 wt. %, between approximately 0.1-1.0 wt. %, between approximately 0.5-5.0 wt. %, between approximately 1.0-5.0 wt. %, between approximately 1.0-2.0 wt. %, between approximately 2.0-5.0 wt. %, between approximately 3.0-5.0 wt. %, or between approximately 4.0-5.0 wt. %, relative to the pharmaceutical composition.

In certain embodiments of the pharmaceutical composition, the ophthalmic device, or the method of treating, disclosed herein, the pharmaceutical composition is an aqueous composition, an ophthalmic formulation, an ophthalmic aqueous formulation, an eye drop formulation, an ocular spray formulation, an ocular pharmaceutical composition contained within a contact lens blister pack, a topical formulation, a topical ophthalmic composition, an ocular gel formulation, an ophthalmic emulsion, ophthalmic liposomes, nano wafers, a nano particle suspension, or an ophthalmic ointment.

In certain embodiments of the pharmaceutical composition, the ophthalmic device, or the method of treating, disclosed herein, the pharmaceutical composition further comprises one or more additional ophthalmically acceptable excipients and additives, comprising carriers, stabilizers, an osmolarity adjusting agent, a preservative, a buffer agent, a tonicity adjusting agent, thickeners, or other excipients.

In certain embodiments of the pharmaceutical composition, the ophthalmic device, or the method of treating, disclosed herein, the pharmaceutical composition is a sustained release formulation or a subconjunctival depot.

In certain embodiments of the pharmaceutical composition, the ophthalmic device, or the method of treating, disclosed herein, the pharmaceutical composition is a sustained release formulation contained within an ophthalmic device.

In certain embodiments of the pharmaceutical composition, the ophthalmic device, or the method of treating, disclosed herein, the ophthalmic device is a contact lens, an ocular insert, a corneal onlay, a corneal inlay, a nano wafer, a liposome, a nanoparticle, a punctal plug, or a hydrogel matrix with microfluid reservoirs.

In certain embodiments of the pharmaceutical composition, the ophthalmic device, or the method of treating, disclosed herein, the ophthalmic device delivers the pharmaceutical composition in a sustained release manner.

In certain embodiments of the pharmaceutical composition, the ophthalmic device, or the method of treating, disclosed herein, the pharmaceutical composition is formulated as an ophthalmic composition, for example, formulated as an ophthalmic composition for treatment of an ophthalmic disorder or condition.

In certain embodiments of the pharmaceutical composition, the ophthalmic device, or the method of treating, disclosed herein, the pharmaceutical composition is formulated as an ophthalmic composition for treatment of pre-myopia, myopia, or progression of myopia.

In certain embodiments of the pharmaceutical composition, the ophthalmic device, or the method of treating, disclosed herein, the pharmaceutical composition is formulated as an ophthalmic composition for treatment of high myopia, moderate myopia, or low myopia.

In certain embodiments of the pharmaceutical composition, the ophthalmic device, or the method of treating, disclosed herein, the pharmaceutical composition is formulated as an ophthalmic composition for treatment of a patient diagnosed as pre-myopic (or at risk of developing myopia).

In certain embodiments of the pharmaceutical composition, the ophthalmic device, or the method of treating, disclosed herein, the pharmaceutical composition is distributed with substantial uniformity throughout the ophthalmic device.

In certain embodiments of the pharmaceutical composition, the ophthalmic device, or the method of treating, disclosed herein, the ophthalmic device is contained within a contact lens blister pack.

In certain embodiments of the pharmaceutical composition, the ophthalmic device, or the method of treating, disclosed herein, the pharmaceutical composition bathes the ophthalmic device within the contact lens blister pack.

In certain embodiments of the pharmaceutical composition, the ophthalmic device, or the method of treating, disclosed herein, the muscarinic antagonist and the adenosine antagonist are co-administered concurrently, co-administered sequentially with administration of the muscarinic antagonist followed by the adenosine antagonist, or co-administered sequentially with administration of the adenosine antagonist followed by the muscarinic antagonist.

In certain embodiments of the pharmaceutical composition, the ophthalmic device, or the method of treating, disclosed herein, the method prevents the progression of myopia in the treated patient.

In certain embodiments of the pharmaceutical composition, the ophthalmic device, or the method of treating, disclosed herein, the method controls the progression of myopia in the treated patient.

In certain embodiments of the pharmaceutical composition, the ophthalmic device, or the method of treating, disclosed herein, the method mitigates the progression of myopia in the treated patient.

In certain embodiments of the pharmaceutical composition, the ophthalmic device, or the method of treating, disclosed herein, the method slows or reduces the progression of myopia in the treated patient.

In certain embodiments of the pharmaceutical composition, the ophthalmic device, or the method of treating, disclosed herein, the method controls, slows, reduces, retards, and/or mitigates, the progression of myopia in the treated patient in the range of between about 5-95%, between about 5-90%, between about 5-80%, between about 5-70%, between about 5-60%, between about 5-50%, between about 5-40%, between about 5-30%, between about 5-20%, between about 10-100%, between about 20-90%, between about 30-90%, between about 40-90%, between about 50-90%, or between about 75-90%, relative to non-treatment.

In certain embodiments, the method of treating myopia in a patient in need thereof, as disclosed herein, may increase choroidal thickness of an eye of the treated patient, for example, increase the choroidal thickness of an eye of the treated patient in the range of between approximately 5-100%, relative to non-treatment, such as increase the choroidal thickness of an eye of the treated patient in the range of between about 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10100%, between approximately 20-90%, between approximately 25-90%, between approximately 30-90%, between approximately 40-90%, between approximately 50-90%, or between approximately 75-90%, relative to non-treatment.

In certain embodiments of the pharmaceutical composition, the ophthalmic device, or the method of treating, disclosed herein, the use of the pharmaceutical composition, the ophthalmic device or the method of treating limits the increase in photopic pupil size of the eye of the user to about 1-2 mm, about 1 mm, about 2 mm, less than 2 mm, less than 1 mm.

In certain embodiments of the pharmaceutical composition, the ophthalmic device, or the method of treating, disclosed herein, the use of the pharmaceutical composition, the ophthalmic device or the method of treating limits the reduction in accommodative amplitude of the eye of the user to about 1.0-6.0D, 1.0-5.0D, 1.0-4.0D, 1.0-3.0D, 1.0-2.0D, less than 6.0D, less than 5.0D, less than 4.0D, less than 3.0D, less than 2.0D and less than 1.0D.

In certain embodiments of the pharmaceutical composition, the ophthalmic device, or the method of treating, disclosed herein, the method reverses the progression of myopia in the treated patient.

In certain embodiments of the pharmaceutical composition, the ophthalmic device, or the method of treating, disclosed herein, the patient suffers from high myopia, moderate myopia, or low myopia.

In certain embodiments of the pharmaceutical composition, the ophthalmic device, or the method of treating, disclosed herein, the patient is pre-myopic (or at risk of developing myopia).

In certain embodiments of the pharmaceutical composition, the ophthalmic device, or the method of treating, disclosed herein, the method increases choroidal thickness of an eye of the treated patient.

In certain embodiments of the pharmaceutical composition, the ophthalmic device, or the method of treating, disclosed herein, the method increases choroidal thickness of an eye of the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 11000%, between approximately 20-90%, between approximately 25-90%, between approximately 30-90%, between approximately 40-90%, between approximately 50-90%, or between approximately 75-90%, relative to non-treatment.

In certain embodiments of the pharmaceutical composition, the ophthalmic device, or the method of treating, disclosed herein, the method prevents, controls, slows, reduces, retards, and/or mitigates, axial (or longitudinal) growth of an eye of the treated patient.

In certain embodiments of the pharmaceutical composition, the ophthalmic device, or the method of treating, disclosed herein, the method controls, slows, reduces, retards, and/or mitigates, the progression of myopia, increases choroidal thickness of an eye (e.g., a myopic eye, a pre-myopic eye, or an eye at risk of developing myopia), and/or reduces axial (or longitudinal) growth of an eye (e.g., a myopic eye, a pre-myopic eye, or an eye at risk of developing myopia) of a patient diagnosed as having myopia or at risk of developing myopia

In certain embodiments of the pharmaceutical composition, the ophthalmic device, or the method of treating, disclosed herein, the method controls, slows, reduces, retards, and/or mitigates, axial (or longitudinal) growth of an eye of the treated patient in between about 5-95%, between about 5-90%, between about 5-80%, between about 5-70%, between about 5-60%, between about 5-50%, between about 5-40%, between about 5-30%, between about 5-20%, between about 10100%, between about 20-90%, between about 30-90%, between about 40-90%, between about 50-90%, or between about 75-90%, relative to non-treatment.

In certain embodiments of the pharmaceutical composition, the ophthalmic device, or the method of treating, disclosed herein, the patient is treated for a period of about between 1 month to 10 years, such as for a period of at least 6 months, at least 1 year, at least 2 years, at least 3 years, at least 5 years, at least 7 years, or at least 9 years.

In certain embodiments of the pharmaceutical composition, the ophthalmic device, or the method of treating, disclosed herein, the method results in less severe adverse side effects, relative to atropine monotherapy.

In certain embodiments of the pharmaceutical composition, the ophthalmic device, or the method of treating, disclosed herein, the method results in a smaller increase of pupil size, relative to atropine monotherapy.

In certain embodiments of the pharmaceutical composition, the ophthalmic device, or the method of treating, disclosed herein, the method results in a smaller decrease in accommodative amplitude, relative to atropine monotherapy.

In certain embodiments of the pharmaceutical composition, the ophthalmic device, or the method of treating, disclosed herein, the pharmaceutical composition is ophthalmically administered to an eye of the patient.

In certain embodiments of the pharmaceutical composition, the ophthalmic device, or the method of treating, disclosed herein, the pharmaceutical composition is topically administered.

In certain embodiments of the pharmaceutical composition, the ophthalmic device, or the method of treating, disclosed herein, the pharmaceutical composition is administered to the eye in the form of an eye drop formulation, an ocular spray formulation, or an ocular gel formulation.

In certain embodiments of the pharmaceutical composition, the ophthalmic device, or the method of treating, disclosed herein, the pharmaceutical composition is administered to the eye in the form of an ophthalmic emulsion, ophthalmic liposomes, nano wafers, a nano particle suspension, or an ophthalmic ointment.

In certain embodiments of the pharmaceutical composition, the ophthalmic device, or the method of treating, disclosed herein, the pharmaceutical composition is ophthalmically administered to an eye of the patient via an ophthalmic device.

In certain embodiments of the pharmaceutical composition, the ophthalmic device, or the method of treating, disclosed herein, the pharmaceutical composition is administered 1, 2, 3, 4, or 5, times per day.

In certain embodiments of the pharmaceutical composition, the ophthalmic device, or the method of treating, disclosed herein, the patient is of an age of about 4-18 years, or of an age of about 16-26 years.

Other features and advantages of the subject matter described herein will be apparent from the description and figures, and from the claims.

### BRIEF DESCRIPTION OF THE DRAWINGS

Aspects of the embodiments described herein may be best understood from the following detailed description when read with the accompanying figures.

FIG. 1 is a flow chart illustrating a procedure for evaluating choroidal thickness changes in a primate resulting from administering an atropine or caffeine monotherapy eye drop formulation or a combination therapy eye drop formulation containing atropine and caffeine.

FIG. 2 is a graph illustrating choroidal thickness measurements resulting from administering to a primate an atropine or caffeine monotherapy eye drop formulation or a combination therapy eye drop formulation containing atropine and caffeine.

FIG. 3 is a flow chart illustrating a procedure for evaluating choroidal thickness changes in a primate resulting from administering an atropine or caffeine monotherapy eye drop formulation or a combination therapy eye drop formulation containing atropine and caffeine.

FIG. 4 is a graph illustrating choroidal thickness measurements resulting from administering to a primate an atropine or caffeine monotherapy eye drop formulation or a combination therapy eye drop formulation containing atropine and caffeine.

FIGS. 5A-5D are graphs illustrating refractive error and axial length changes from administering to a primate either an atropine eye drop alone or an eyedrop formulation containing atropine and caffeine.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Myopia, an axial elongation of the eye, affects a large proportion of the population. The onset of myopia is generally during the grade school years and progresses until growth of the eye is completed. Myopia progression can lead to increasing visual impairment despite the use of corrective lenses. The present disclosure recognizes the importance of compositions and treatments for treating, preventing, controlling, slowing, reducing, retarding, and/or mitigating, the development or progression of myopia, especially with pharmaceutical compositions, ophthalmic devices containing or delivering the same, and methods of using the same, that are conveniently administered or conducted, that reduce potential side effects, and provide therapeutic benefits, or combinations thereof.

Drug combination therapy is a widely used and powerful strategy in medicine with the aim to achieve a synergistic therapeutic effect, dose and toxicity reduction, and to minimize or delay the induction of drug resistance (Chou T C., “Drug combination studies and their synergy quantification using the Chou-Talalay method,” Cancer Res. (2010) 70:440-6). The present disclosure identifies certain compounds that provide a synergistic effect with muscarinic receptor antagonists, such as atropine, to enhance the myopia reduction or myopia slowing effect while avoiding or minimizing adverse side effects, such as those observed with atropine monotherapy.

The present application provides a pharmaceutical composition of a non-selective muscarinic receptor antagonist and a non-selective adenosine antagonist for topical or ocular application, and ophthalmic devices containing or delivering the same, and methods of using the same, for controlling and/or reducing the progression of myopia.

In certain embodiments, the pharmaceutical composition may comprise or consist of a muscarinic receptor antagonist and an adenosine receptor antagonist. In certain embodiments, the muscarinic antagonist may be a nonselective muscarinic acetylcholinergic receptor antagonist, or may be an M1 selective antagonist. In certain embodiments, the muscarinic antagonist may be a non-selective muscarinic receptor antagonist, such as a non-selective muscarinic receptor antagonist in low concentrations. In certain embodiments, the adenosine antagonist is a non-selective adenosine antagonist.

In certain embodiments, the muscarinic antagonist provided with the pharmaceutical composition disclosed herein may be atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolomine (L-hyoscine), hydroxyzine, ipratropium, tropicamide, cyclopentolate, pirenzepine, homatropine, solifenacin, darifenacin, benzatropine, oxyphenonium, mebeverine, procyclidine, aclidinium bromide, trihexyphenidylbenzhexol, tolterodine, or a pharmaceutically acceptable salt thereof. In preferred embodiments, the muscarinic receptor antagonist is atropine, or a pharmaceutically acceptable salt thereof. In certain embodiments, the muscarinic receptor antagonist is tropine, or a pharmaceutically acceptable salt thereof. In certain embodiments, the muscarinic receptor antagonist is tropic acid. In certain embodiments, the muscarinic receptor antagonist provided with the pharmaceutical composition disclosed herein may be present in low concentrations, for example, in an amount of less than 0.05 wt. %, such as in an amount of 0.045 wt. % or less, 0.04 wt. % or less, 0.035 wt. % or less, or 0.03 wt. % or less, relative to the pharmaceutical composition. For example, in certain embodiments, the muscarinic receptor antagonist provided with the pharmaceutical composition disclosed herein may be present in low concentrations, for example, in the range of between approximately 0.001 to less than 0.05 wt. %, such as, between approximately 0.001-0.045 wt. %, between approximately 0.001-0.04 wt. %, between approximately 0.001-0.035 wt. %, between approximately 0.001-0.03 wt. %, between approximately 0.001-0.025 wt. %, between approximately 0.001-0.02 wt. %, between approximately 0.001-0.015 wt. %, between approximately 0.001-0.01 wt. %, between approximately 0.001-0.005 wt. %, between approximately 0.005-0.03 wt. %, between approximately 0.005-0.04 wt. %, between approximately 0.01-0.03 wt. %, between approximately 0.01-0.045 wt. %, between approximately 0.01-0.04 wt. %, between approximately 0.02-0.04 wt. %, between approximately 0.02-0.03 wt. %, between approximately 0.015-0.025 wt. %, between approximately 0.015-0.03 wt. %, or between approximately 0.015-0.035 wt. %, relative to the pharmaceutical composition.

In certain embodiments, the muscarinic antagonist may be present in the pharmaceutical composition disclosed herein in an amount of less than 0.05 wt. %, such as in an amount of approximately 0.001 wt %, approximately 0.002 wt. %, approximately 0.005 wt. %, approximately 0.01 wt. %, approximately 0.015 wt. %, approximately 0.02 wt. %, approximately 0.025 wt. %, approximately 0.03 wt. %, approximately 0.035 wt. %, approximately 0.04 wt. %, or approximately 0.045 wt. %, relative to the pharmaceutical composition.

In certain embodiments, the adenosine receptor antagonist provided with the pharmaceutical composition disclosed herein may be a non-selective adenosine antagonist. For example, in certain embodiments, the non-selective adenosine antagonist may be a xanthine derivative, such as a substituted xanthine derivative, or a pharmaceutically acceptable salt thereof, such as caffeine; 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); 1,7-dimethylxanthine; or a substituted xanthine detailed in U.S. Pat. No. 6,710,051; or mixtures thereof. In preferred embodiments, the adenosine receptor antagonist is caffeine or 7-methylxanthine, or a pharmaceutically acceptable salt thereof, for example, is caffeine, or a pharmaceutically acceptable salt thereof, such as caffeine citrate. In certain embodiments, the adenosine antagonist provided with the pharmaceutical composition disclosed herein may be present in an amount in the range of between approximately 0.1-5.0 wt. %, relative to the pharmaceutical composition, such as present in an amount in the range of between approximately 0.1-4.0 wt. %, between approximately 0.1-3.0 wt. %, between approximately 0.1-2.0 wt. %, between approximately 0.1-1.0 wt. %, between approximately 0.5-5.0 wt. %, between approximately 1.0-5.0 wt. %, between approximately 1.0-2.0 wt. %, between approximately 2.0-5.0 wt. %, between approximately 3.0-5.0 wt. %, or between approximately 4.0-5.0 wt. %, relative to the pharmaceutical composition.

In certain embodiments of the pharmaceutical composition, the ophthalmic device, or the method of treating, disclosed herein, the use of the pharmaceutical composition, the use of the ophthalmic device, or the method of treating controls, slows, reduces, retards, and/or mitigates, the progression of myopia in the treated patient in the range of between about 5-95%, between about 5-90%, between about 5-80%, between about 5-70%, between about 5-60%, between about 5-50%, between about 5-40%, between about 5-30%, between about 5-20%, between about 10-100%, between about 20-90%, between about 30-90%, between about 40-90%, between about 50-90%, or between about 75-90%, relative to non-treatment.

In certain embodiments of the pharmaceutical composition, the ophthalmic device, or the method of treating, disclosed herein, the use of the pharmaceutical composition, the use of the ophthalmic device, or the method of treating increases the size of the photopic pupil of an eye of the user to about 1 mm to 2 mm, about 1 mm, about 2 mm, less than 2 mm or less than 1 mm.

In certain embodiments of the pharmaceutical composition, the ophthalmic device, or the method of treating, disclosed herein, the reduction in the accommodative amplitude with the pharmaceutical composition, the ophthalmic device or the method of treating is about 1.0-6.0D, 1.0-5.0D, 1.0-4.0D, 1.0-3.0D, 1.0-2.0D, less than 6.0D, less than 5.0D, less than 4.0D, less than 3.0D, less than 2.0D and less than 1.0D.

In certain embodiments, the pharmaceutical composition may comprise a “hybrid molecule” (sometimes referred to herein as a “conjugate molecule” or “conjugate compound”) synthesized from a muscarinic receptor antagonist and an adenosine receptor antagonist. In certain embodiments, the pharmaceutical composition may comprise a hybrid molecule synthesized from atropine and caffeine. In certain embodiment, the pharmaceutical composition may comprise a hybrid molecule comprising one molecule of atropine conjugated with one molecule of caffeine, such as a conjugate compound of Formula (I) (having one molecule of atropine conjugated to the N1 position of caffeine). a conjugate compound of Formula (II) (having one molecule of atropine conjugated to the N3 position of caffeine), or a conjugate compound of Formula (III) (having one molecule of atropine conjugated to the N7 position of caffeine).

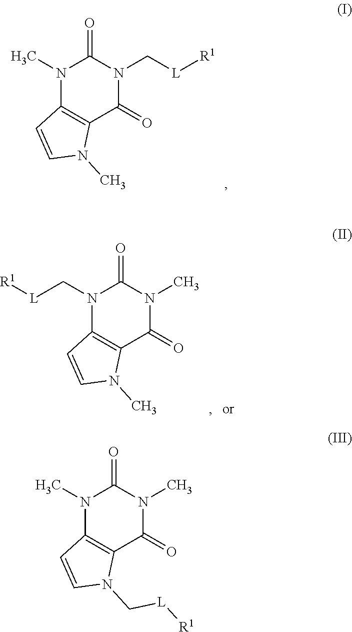

wherein R¹ is an atropine moiety, and wherein L is a divalent linker, such that the divalent linker group covalently conjugates an atropine molecule with a caffeine molecule. In certain embodiments, suitable divalent linkers may include a hydrocarbon linker comprising stable bonds, such as hydrocarbon linker that are hydrophobic. Suitable hydrocarbon linkers may include a polyalkyl linker, e.g., C₅-C₂₀ alkyl linker; a C₅-C₆ cycloalkyl linker, e.g., 1,4-cyclohexyl linker, 1,3-cyclohexyl linker, 1,2-cyclohexyl linker, 1,3-cyclopentyl, or 1,2-cyclopentyl; a C₅-C₆ cycloalkenyl linker. In certain embodiments, suitable divalent linkers may include stable bonds that are hydrophilic, such as a polyethylene glycol linker, e.g., —(OCH₂CH₂)ₙ—, wherein n is 5-20. In certain embodiments, suitable divalent linkers may include ester linkages susceptible to hydrolysis by esterases, such as an acetyl linker, e.g., —(O(CO)CH₂)—. For example, by way of illustration, the hybrid molecule may be a conjugate compound having Formula (I) having one molecule of atropine conjugated from the N-methyl group to the N1 position of caffeine via an L divalent linker that is a polyalkyl linker (wherein n is 5-20), via an L divalent linker that is a polyethylene glycol linker (wherein n is 5-20), or via an L divalent linker that is an acetyl linker.

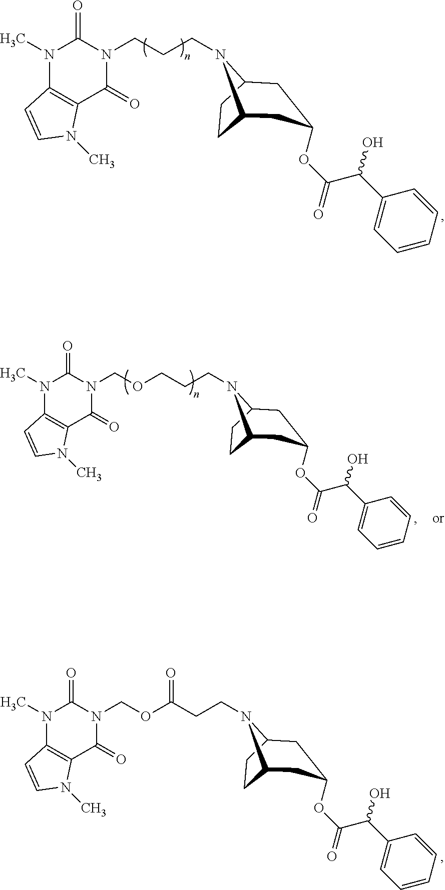

respectively. In certain embodiments, the hybrid molecule is a conjugate compound having Formula (II) or (III) having one molecule of atropine conjugated from the N-methyl group to the N3 or N7 position of caffeine, respectively, via an L divalent linker that is a polyalkyl linker (wherein n is 5-20), via an L divalent linker that is a polyethylene glycol linker (wherein n is 5-20), or via an L divalent linker that is an acetyl linker.

In certain embodiments, the pharmaceutical composition may comprise a hybrid molecule comprising one molecule of atropine conjugated to two molecules of caffeine, such as a conjugate compound of Formula (IV):

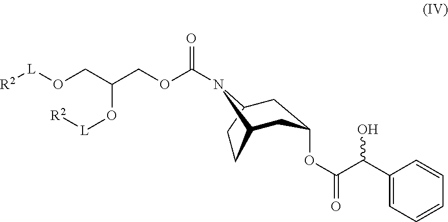

wherein an N-carbamate derivative of atropine is conjugated via a trivalent linker, such as a 1,2,3-propane triol moiety, to two divalent linkers (L), wherein L may independently be a polyethylene glycol linker (wherein n is independently 5-20), a polyalkyl linker, e.g., C₅-C₂₀ alkyl linker, a C₅-C₆ cycloalkyl linker, a C₅-C₆ cycloalkenyl linker; or an ester linkage, such as an acetyl linker, e.g., —(O(CO)CH₂)—, and wherein each of the two independent divalent linkers are further conjugated to R² groups, wherein R² is independently a caffeine moiety independently conjugated via the N-methyl group at the N1, N3, or N7 position. For example, by way of illustration, the hybrid molecule may be a conjugate compound having Formula (IV) having an N-carbamate derivative of atropine conjugated via a 1,2,3-propane triol to two independent divalent linkers (L) that are polyethylene glycol linkers (wherein n is independently 5-20), and wherein R² is independently a caffeine moiety independently conjugated via the N-methyl group at the N1 position:

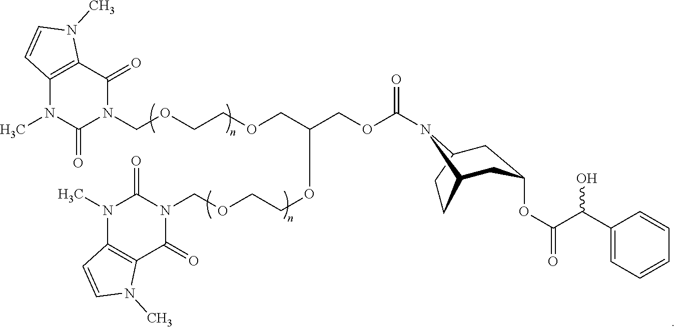

In certain embodiments, the hybrid molecule is a conjugate compound having Formula (TV) having an N-carbamate derivative of atropine conjugated via a 1,2,3-propane triol to two independent divalent linkers (L) that are polyethylene glycol linkers (wherein n is independently 5-20), and wherein R² is independently a caffeine moiety independently conjugated via the N-methyl group at the N3 or N7 position of caffeine.

In certain embodiments, the pharmaceutical composition may comprise a hybrid molecule comprising two molecules of atropine conjugated to one molecule of caffeine, such as a conjugate compound of Formula (V):

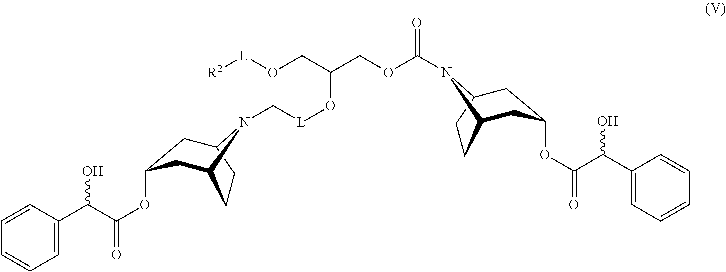

wherein an N-carbamate derivative of atropine is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker, a C₅-C₆ cycloalkyl linker, a C₅-C₆ cycloalkenyl linker, or an ester linkage, such as an acetyl linker, e.g., —(O(CO)CH₂)—, wherein one of the independent divalent linkers are further conjugated to an atropine moiety via the N-methyl group, and wherein one of the independent divalent linkers are further conjugated to an R² group, wherein R² is a caffeine moiety conjugated via the N-methyl group at the N1, N3, or N7 position. For example, by way of illustration, the hybrid molecule may be a conjugate compound having Formula (V) having an N-carbamate derivative of atropine conjugated via a 1,2,3-propane triol to two independent divalent linkers (L) that are polyethylene glycol linkers (wherein n is independently 5-20), wherein one of the independent divalent linkers are further conjugated to an atropine moiety via the N-methyl group, and wherein one of the independent divalent linkers are further conjugated to an R² group, and wherein R² is independently a caffeine moiety independently conjugated via the N-methyl group at the N1 position:

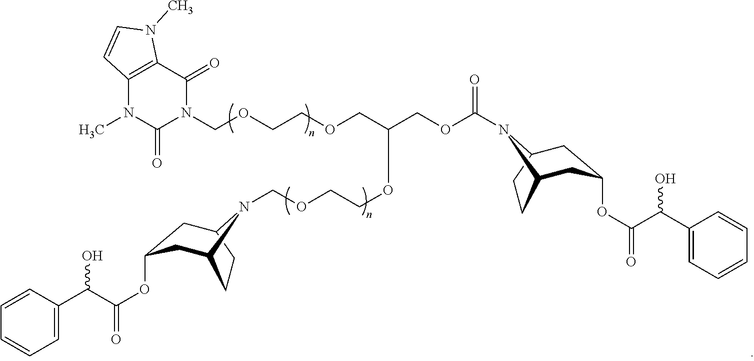

In certain embodiments, the hybrid molecule is a conjugate compound having Formula (V) having an N-carbamate derivative of atropine conjugated via a 1,2,3-propane triol to two independent divalent linkers (L) that are polyethylene glycol linkers (wherein n is independently 5-20), wherein one of the independent divalent linkers are further conjugated to an atropine moiety via the N-methyl group, and wherein one of the independent divalent linkers are further conjugated to an R² group, and wherein R² is independently a caffeine moiety independently conjugated via the N-methyl group at the N3 or N7 position of caffeine.

In certain embodiments, the pharmaceutical composition may comprise a hybrid molecule comprising one molecule of tropine conjugated with one molecule of caffeine, such as a conjugate compound of Formula (VI):

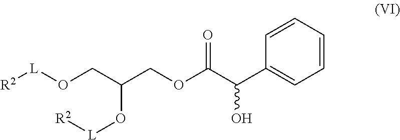

wherein a tropine moiety is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker; a C₅-C₆ cycloalkyl linker; a C₅-C₆ cycloalkenyl linker; or an ester linkage, such as an acetyl linker, e.g., —(O(CO)CH₂)—, and wherein each of the two independent divalent linkers are further conjugated to R² groups, wherein R² is independently a caffeine moiety independently conjugated via the N-methyl group at the N1, N3, or N7 position. For example, by way of illustration, the hybrid molecule may be a conjugate compound having Formula (VI) having a tropine moiety conjugated via a 1,2,3-propane triol to two independent divalent linkers (L) that are polyethylene glycol linkers (wherein n is independently 5-20), and wherein R² is independently a caffeine moiety independently conjugated via the N-methyl group at the N1 position:

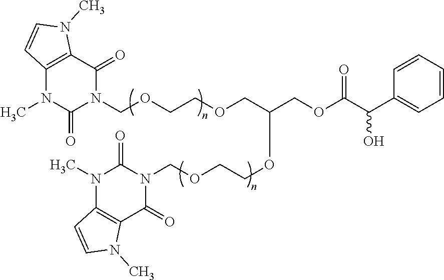

In certain embodiments, the hybrid molecule is a conjugate compound having Formula (V) having a tropine moiety conjugated via a 1,2,3-propane triol to two independent divalent linkers (L) that are polyethylene glycol linkers (wherein n is independently 5-20), and wherein R² is independently a caffeine moiety independently conjugated via the N-methyl group at the N3 or N7 position of caffeine.

In certain embodiments, the pharmaceutical composition may comprise a hybrid molecule comprising two molecules of tropine conjugated to one molecule of caffeine, such as a conjugate compound of Formula (VII):

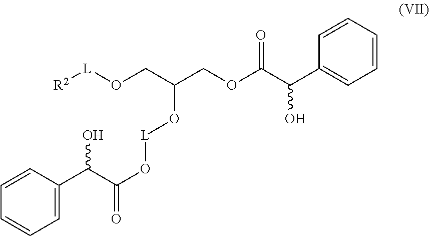

wherein a tropine moiety is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker; a C₅-C₆ cycloalkyl linker; a C₅-C₆ cycloalkenyl linker; or an ester linkage, such as an acetyl linker, e g., —(O(CO)CH₂)—, wherein one of the independent divalent linkers are further conjugated to a tropine moiety, and wherein one of the independent divalent linkers are further conjugated to an R² group, wherein R² is a caffeine moiety conjugated via the N-methyl group at the N1, N3, or N7 position. For example, by way of illustration, the hybrid molecule may be a conjugate compound having Formula (VII) having a tropine moiety conjugated via a 1,2,3-propane trio to two independent divalent linkers (L) that are polyethylene glycol linkers (wherein n is independently 5-20), wherein one of the independent divalent linkers are further conjugated to a tropine moiety, and wherein one of the independent divalent linkers are further conjugated to an R² group, and wherein R² is independently a caffeine moiety independently conjugated via the N-methyl group at the N1 position:

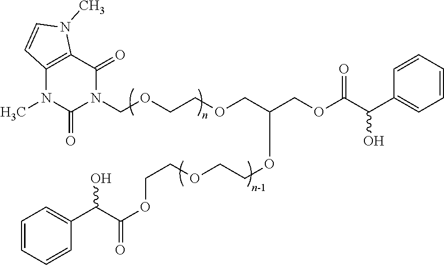

In certain embodiments, the hybrid molecule is a conjugate compound having Formula (VII) having a tropine conjugated via a 1,2,3-propane triol to two independent divalent linkers (L) that are polyethylene glycol linkers (wherein n is independently 5-20), wherein one of the independent divalent linkers are further conjugated to a tropine moiety, and wherein one of the independent divalent linkers are further conjugated to an R² group, and wherein R² is independently a caffeine moiety independently conjugated via the N-methyl group at the N3 or N7 position of caffeine.

In certain embodiments, the pharmaceutical composition comprising or consisting of a muscarinic antagonist and an adenosine antagonist may be in the form of an aqueous composition, an ophthalmic formulation, an ophthalmic aqueous formulation, an eye drop formulation, an ocular spray formulation, an ocular pharmaceutical composition contained within a contact lens blister pack, a topical formulation, a topical ophthalmic composition, an ocular gel formulation, an ophthalmic emulsion, ophthalmic liposomes, nano wafers, a nano particle suspension, or an ophthalmic ointment.

In certain embodiments, the pharmaceutical composition, as disclosed herein, may be an ophthalmic aqueous formulation, such as in the form of eye drops. For example, the ophthalmic aqueous formulation, as described herein, may be packaged in an eye drop bottle and administered as drops. In certain embodiments, the ophthalmic aqueous formulation may be administered as a single administration (i.e., a single dose), which may include a single drop, two drops, three drops or more into the eyes of the patient. In certain embodiments, one dose of the ophthalmic aqueous formulation described herein is one drop of the aqueous composition from the eye drop bottle.

In certain embodiments, the pharmaceutical composition, as disclosed herein, may be an ophthalmic gel formulation. For example, the ophthalmic gel formulation may be packaged in an eye drop bottle and administered as drops. In certain embodiments, the ophthalmic gel formulation may be administered as a single administration (i.e., a single dose), which may include a single drop, two drops, three drops or more into the eyes of the patient. In certain embodiments, one dose of the ophthalmic gel described herein is one drop of the gel composition from the eye drop bottle.

In certain embodiments, the pharmaceutical composition, as disclosed herein, may be an ophthalmic ointment formulation. For example, the ophthalmic ointment formulation may be packaged in tubes or other squeezable containers with a dispensing nozzle through which strips of the ointment are delivered. In certain embodiments, the ophthalmic ointment formulation may be administered as a single administration (i.e., a single dose), which may include a single strip, or multiple strips into the eyes of the patient. In certain embodiments, one dose of the ophthalmic ointment is one strip of the ointment composition dispensed through the nozzle of a dispersing tube.

In certain embodiments, the pharmaceutical composition comprising or consisting of a muscarinic antagonist and an adenosine antagonist may further comprise one or more additional ophthalmically acceptable excipients and additives, comprising for example, carriers, stabilizers, osmolarity adjusting agent, a preservative, a buffer agent, or a tonicity adjusting agent, thickeners and other excipients.

Carriers used in certain embodiments are typically suitable for topical administration and may comprise water, mixtures of water and water-miscible solvents such as C to C₇-alkanols, vegetable or mineral oils comprising from 0.1 to 5% by weight hydroxyethylcellulose, ethyl oleate, carboxymethylocelluse and other water soluble polymers for ophthalmic use such as carboxy methycellulose, hydroxymethylcellolose, hydroxyethylcellulose, ethyl acrulate, polyacrylamide, natural products such as pectin, alginates, starch derivatives and also other synthetic products, such as polyvinyl alcohol, polyvinylpyrrolidone, polyvinyl methyl ether, polyethylene oxide, cross-linked polyacrylic acid, such as neutral Carbopol, or mixtures of those polymers; naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate. For example, in certain embodiments, the pharmaceutical composition comprising or consisting of a muscarinic antagonist and an adenosine antagonist may further comprise an osmolarity adjusting agent as an additional ophthalmically acceptable agent, such as sodium chloride. In certain embodiments, the additional ophthalmically acceptable agent contained with the pharmaceutical composition disclosed herein may be a preservative, such as benzalkonium chloride, cetrimonium, sodium perborate, stabilized oxychloro complex, SofZia, polyquaternium-1, chlorobutanol, edetate disodium, polyhexamethylene biguanide, or combinations thereof. In certain embodiments, the additional ophthalmically acceptable agent contained with the pharmaceutical composition disclosed herein may be a buffer agent, such as a borate, a borate-polyol complex, a phosphate buffering agent, a citrate buffering agent, an acetate buffering agent, a carbonate buffering agent, an organic buffering agent, an amino acid buffering agent, or combinations thereof. In certain embodiments, the additional ophthalmically acceptable agent contained with the pharmaceutical composition disclosed herein may be a tonicity adjusting agent, such as sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In certain embodiments, the pharmaceutical composition comprising or consisting of a muscarinic antagonist and an adenosine antagonist may be in the form of a sustained release formulation, such as a sustained release formulation contained within an ophthalmic device, or a subconjunctival depot. For example, in certain embodiments, the pharmaceutical composition comprising or consisting of a muscarinic antagonist and an adenosine antagonist is a sustained release formulation contained within an ophthalmic device, wherein the ophthalmic device may be a contact lens, an ocular insert, a corneal onlay, a corneal inlay, a nano wafer, a liposome, a nanoparticle, a punctal plug, or a hydrogel matrix with microfluid reservoir. The sustained release formulation of the pharmaceutical composition disclosed herein, when contained in an ophthalmic device, is delivered from the ophthalmic device in a sustained release manner. In certain embodiments, the pharmaceutical composition, such as an ophthalmic composition, comprising or consisting of a muscarinic antagonist and an adenosine antagonist, may be distributed with substantial uniformity (e.g., at least 50% uniformity, such as between 80-95% uniformity) throughout the ophthalmic device.

In certain embodiments, the pharmaceutical composition comprising or consisting of a muscarinic antagonist and an adenosine antagonist may be formulated as an ophthalmic composition, for example, formulated as an ophthalmic composition for treatment of an ophthalmic disorder or condition, such as for the treatment of pre-myopia, myopia, or progression of myopia. In certain embodiments, the pharmaceutical composition comprising or consisting of a muscarinic antagonist and an adenosine antagonist may be formulated as an ophthalmic composition for treatment of high myopia (myopia of greater than −5.00 diopters (D) (i.e., more negative and further from 0.00 diopeters), such as greater than −6.00 diopters). In certain embodiments, the pharmaceutical composition comprising or consisting of a muscarinic antagonist and an adenosine antagonist may be formulated as an ophthalmic composition for treatment of moderate myopia (myopia in the range of between about −3.00 diopters to about −5.00 diopters). In certain embodiments, the pharmaceutical composition comprising or consisting of a muscarinic antagonist and an adenosine antagonist may be formulated as an ophthalmic composition for treatment of low myopia (myopia of −3.00 diopters or less, i.e., closer to 0.00 diopters). In certain embodiments, the pharmaceutical composition comprising or consisting of a muscarinic antagonist and an adenosine antagonist may be formulated as an ophthalmic composition for treatment of a patient diagnosed as pre-myopic (or at risk of developing myopia).

The present application further provides a method treating myopia in a patient in need thereof, comprising administering a pharmaceutical composition (as disclosed herein) containing a muscarinic antagonist and an adenosine antagonist. In certain embodiments, the method of treatment disclosed herein prevents, controls, slows, reduces, retards, and/or mitigates, the progression of myopia in the treated patient, such as prevents or controls the progression of myopia in the treated patient. For example, in certain embodiments, the method of treatment disclosed herein controls, slows, reduces, retards, and/or mitigates, the progression of myopia in the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between approximately 50-90%, or between approximately 75-90%, relative to non-treatment. In certain embodiments, the method of treatment disclosed herein prevents and/or reverses the progression of myopia in the treated patient. The patient suffering from myopia may be at risk of developing myopia (e.g., is pre-myopic) or suffer from high myopia, moderate myopia, or low myopia. In certain embodiments, the method of treating myopia in a patient in need thereof, as disclosed herein, the patient is of an age of about 4-18 years, or of an age of about 16-26 years.

In certain embodiments, the method of treating myopia in a patient in need thereof, as disclosed herein, may increase choroidal thickness of an eye of the treated patient, for example, increase the choroidal thickness of an eye of the treated patient in the range of between approximately 5-100%, relative to non-treatment, such as increase the choroidal thickness of an eye of the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between approximately 50-90%, or between approximately 75-90%, relative to non-treatment.

In certain embodiments, the method of treating myopia in a patient in need thereof, as disclosed herein, may control, slow, reduce, retard, and/or mitigate, axial (or longitudinal) growth of an eye of the treated patient, for example, control, slow, reduce, retard, and/or mitigate, axial (or longitudinal) growth of an eye of the treated patient in the range of between approximately 5-100%, relative to non-treatment, such as control, slow, reduce, retard, and/or mitigate, axial (or longitudinal) growth of an eye of the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between approximately 50-90%, or between approximately 75-90%, relative to non-treatment.

In certain embodiments, the method of treating myopia in a patient in need thereof, as disclosed herein, may comprise administering a pharmaceutical composition comprising a muscarinic antagonist, and administering a pharmaceutical composition comprising an adenosine antagonist. For example, the method of treating may involve co-administering the muscarinic antagonist and the adenosine antagonist as separate pharmaceutical compositions (or agents) rather than in a single combined pharmaceutical composition. In certain embodiments, the method of treating myopia in a patient in need thereof may comprise co-administering a pharmaceutical composition comprising a muscarinic antagonist concurrently with a pharmaceutical composition comprising an adenosine antagonist, or co-administering sequentially (administering the muscarinic antagonist followed by the adenosine antagonist, or administering the adenosine antagonist followed by the muscarinic antagonist). In certain embodiments, the method of treating myopia in a patient in need thereof may comprise administering a hybrid molecule that comprises a muscarinic antagonist conjugated with an adenosine antagonist. In certain embodiments, the method of treating myopia in a patient in need thereof may comprise administering a hybrid molecule that comprises one or more molecules of a muscarinic antagonist conjugated with one or more molecules of an adenosine antagonist.

In certain embodiments, the method of treating myopia in a patient in need thereof, as disclosed herein, may involve treating the patient for a period of between about 1 month to 10 years, for example, for a period of at least 6 months, at least 1 year, at least 2 years, at least 3 years, at least 5 years, at least 7 years, or at least 9 years.

In certain embodiments, the method of treating myopia in a patient in need thereof, as disclosed herein, may result in less severe adverse side effects, relative to atropine monotherapy. For example, the method of treating myopia with the pharmaceutical composition comprising a muscarinic antagonist and an adenosine antagonist, as disclosed herein, may result in the treated patient having a smaller increase of pupil size, relative to atropine monotherapy. In certain embodiments, the method of treating myopia with the pharmaceutical composition comprising a muscarinic antagonist and an adenosine antagonist, as disclosed herein, may result in the treated patient having a smaller decrease in accommodative amplitude, relative to atropine monotherapy.

In certain embodiments, the method of treating myopia in a patient in need thereof, as disclosed herein, the pharmaceutical composition may be ophthalmically administered directly to an eye of the patient, or may be topically administered to the patient. For example, in certain embodiments, the pharmaceutical composition may be administered to the eye in the form of an eye drop formulation, an ocular spray formulation, an ocular gel formulation, an ophthalmic emulsion, ophthalmic liposomes, nano wafers, a nano particle suspension, or an ophthalmic ointment, according to the method of treating myopia, as disclosed herein. For example, in certain embodiments, the pharmaceutical composition may be ophthalmically administered to an eye of the patient via an ophthalmic device, according to the method of treating myopia, as disclosed herein, wherein the ophthalmic device may be a contact lens, an ocular insert, a corneal onlay, a corneal inlay, a nano wafer, a liposome, a nanoparticle, a punctal plug, or a hydrogel matrix with microfluid reservoir. In certain embodiments, the pharmaceutical composition may be administered from the ophthalmic device in a sustained release manner.

In certain embodiments, the method of treating myopia in a patient in need thereof, as disclosed herein, the pharmaceutical composition may be administered 1, 2, 3, 4, or 5, times per day, for example 1-3 times per day, such as once per day.

### EXAMPLES

The following eye drop formulations were used in a primate eyes (animal 656 and animal 659, corresponding to Examples 1 and 2, respectively) to demonstrate changes in choroidal thickness: 0.1 wt. % atropine monotherapy (0.1 wt. % atropine in sterile aqueous 0.3 wt. % hydroxyl-propyl methyl cellulose (“HPMC”), 1.4 wt. % caffeine monotherapy (1.4 wt. % caffeine citrate in sterile aqueous 0.3 wt. % HPMC), and 0.1 wt. % atropine/1.4 wt. % caffeine combination therapy (0.1 wt. % atropine and 1.4 wt. % caffeine citrate solution in sterile aqueous 0.3 wt. % HPMC.

Regarding the measurement of changes in choroidal thickness, it is noted that increases in the choroidal thickness have been utilized as an indicator on the degree of effectiveness in influencing ocular growth changes, suggesting a possible correlation to evaluating effectiveness in treating myopia.

### Example 1

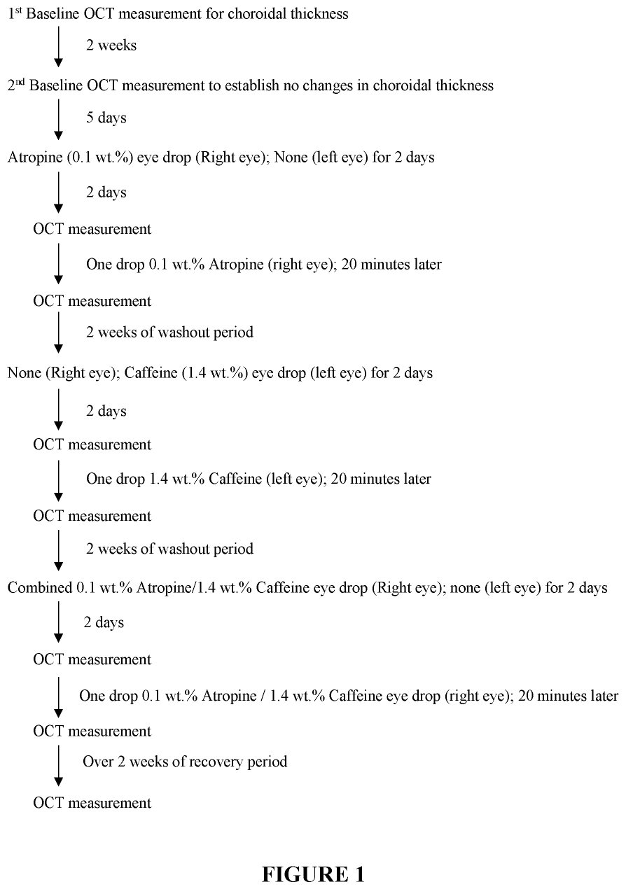

As outlined in FIG. 1, in this experiment, the baseline choroidal thickness of primate 656 was measured via Ocular Coherence Tomography (OCT) in both eyes at baseline 1 (B1) and was repeated 2 weeks later (B2). Beginning on Day 1 (five days after the second baseline measurement), a single drop of 0.1 wt. % atropine monotherapy was instilled in the right eye only once per day for 2 days. On Day 3, a choroidal thickness measurement via OCT was then conducted for both eyes and another drop of 0.1 wt. % atropine monotherapy was instilled in the right eye only and the choroidal thickness measured again in both eyes. Following a washout period of 2 weeks (i.e., beginning on Day 18), a single drop of 1.4 wt. % caffeine monotherapy was instilled in the left eye only once per day for 2 days. On Day 20, the choroidal thickness of both eyes was measured using OCT, another drop of 1.4 wt. % caffeine monotherapy was instilled in the left eye only, and the choroidal thickness was then measured again in both eyes 20 minutes later. This was then followed by a second washout period for 2 weeks, and beginning on Day 35, 0.1 wt. % atropine/1.4 wt. % caffeine combination therapy was instilled in the right eye only once per day for 2 days. On Day 37, the choroidal thickness of both eyes was measured via OCT, another drop of the 0.1 wt. % atropine/1.4 wt. % caffeine combination therapy was instilled in the right eye only, and the choroidal thickness was then measured again in both eyes 20 minutes later. Following the final measurement on Day 37, no drops were instilled for over 2 weeks for a Recovery Period, after which the choroidal thickness of both eyes was again measured on Day 57. The sequence of events outlined in the procedure illustrated in FIG. 1 is also detailed in Table 1.

TABLE 1

| Day | Left Eye | Right Eye |
|---|---|---|
| Baseline | OCT measured | OCT measured |
| 1 (B1) | | |
| Baseline | OCT measured | OCT measured |
| 2 (B2) | | |
| 1 | No drug | 0.1 wt. % atropine monotherapy |
| 2 | No drug | 0.1 wt. % atropine monotherapy |
| 3 | OCT measured, | OCT measured, then 0.1 wt. % |
| | then no drug; | atropine monotherapy; |
| | OCT measured | OCT measured 20 min. later |
| | 20 min. later | |
| 4-17 | No drug | No drug |
| (No drug) | | |
| 18 | 1.4 wt. % caffeine | No drug |
| | monotherapy | |
| 19 | 1.4 wt. % caffeine | No drug |
| | monotherapy | |
| 20 | OCT measured, | OCT measured, then no drug; |
| | then 1.4 wt. % | OCT measured 20 min. later |
| | caffeine | |
| | monotherapy; | |
| | OCT measured | |
| | 20 min. later | |
| 21-34 | No drug | No drug |
| (No drug) | | |
| 35 | No drug | 0.1 wt. % atropine/1.4 wt. % |
| | | caffeine combination therapy |
| 36 | No drug | 0.1 wt. % atropine/1.4 wt. % |
| | | caffeine combination therapy |
| 37 | OCT measured, | OCT measured, then 0.1 wt. % |
| | then no drug; | atropine/1.4 wt. % caffeine |
| | OCT measured | combination therapy; |
| | 20 min. later | OCT measured 20 min. later |
| 38-56 | No drug | No drug |
| (Recovery | | |
| Period) | | |
| 57 | OCT measured | OCT measured |

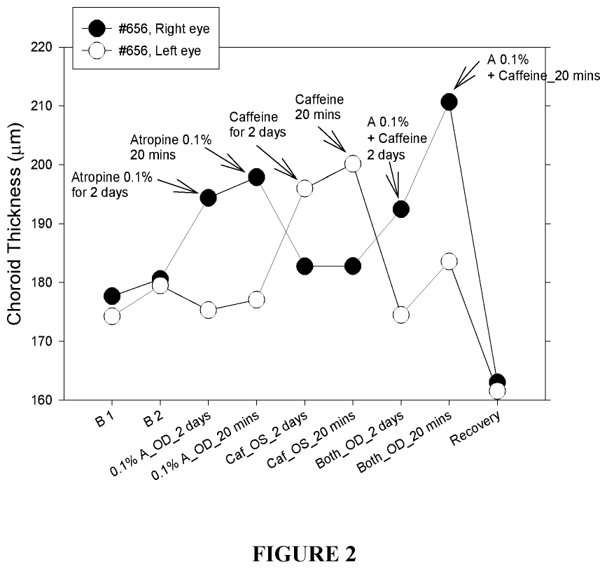

The choroidal thickness results measured during this experiment are shown in FIG. 2; wherein OS refers to left eye (shown as empty circles), and OD refers to right eye (shown as filled-in circles). Choroidal thickness measurements at baseline 1 and 2 of both eyes ranged approximately from about 174 μm to about 180 μm, and was similar between right and left eyes. Use of 0.1 wt. % atropine monotherapy, once per day for 2 days in the right eye only, resulted in an increase in choroidal thickness (increased to about 195 to 198 μm), and similarly use of 1.4 wt. % caffeine monotherapy in the left eye only, once per day for 2 days, resulted in an increase in choroidal thickness. Discontinuation of the use of the 0.1 wt. % atropine monotherapy or the 1.4 wt. % caffeine monotherapy resulted in a decrease in choroidal thickness. Use of a drop of the 0.1 wt. % atropine/1.4 wt. % caffeine combination therapy, once per day for 2 days in the right eye only, also increased the choroidal thickness. The increase in choroidal thickness observed 20 minutes after instillation of a single drop of the 0.1 wt. % atropine/1.4 wt. % caffeine combination therapy was greater than the increase in choroidal thickness observed 20 minutes after the instillation of a single drop of either the 0.1 wt. % atropine monotherapy or the 1.4 wt. % caffeine monotherapy, and significantly, was also greater than additive increase in choroidal thickness observed 20 minutes after the instillation of a single drop of the 0.1 wt. % atropine monotherapy combined with the 1.4 wt. % caffeine monotherapy. The choroidal thickness results measured during this experiment that are shown in FIG. 2, are also provided in Table 2.

TABLE 2

| | Approximate Choroidal | |
|---|---|---|
| | Thickness (μm) | |
| Day | Left Eye | Right Eye |
| Baseline 1 (B1) | 174 | 178 |
| Baseline 2 (B2) | 179 | 180 |
| 3 | 175, 176 | 195, 198 |
| 20 | 196, 200 | 182, 182 |
| 37 | 174, 183 | 192, 211 |
| 57 | 161 | 162 |

This data suggests that administering a combination of a muscarinic antagonist, such as atropine, in combination with an adenosine antagonist, such as caffeine, provides a synergistic effect of increasing choroidal thickness as determined by OCT, relative to the increase in choroidal thickness achieved by either monotherapy. Although adverse effects result from the use of atropine at 0.1 wt. % concentration (data not shown here), this data shows that addition of an adenosine antagonist, such as caffeine, does not diminish the activity of a muscarinic antagonist, such as atropine, and that the addition of the adenosine antagonist may improve upon the efficacy of the muscarinic antagonist. Moreover, this data also suggests that combining an adenosine antagonist with a muscarinic antagonist may provide a pathway to mitigate the adverse side effects associated with muscarinic antagonist monotherapy, such that the adverse side effects may be minimized or reduced, while simultaneously increasing or maintaining the effectiveness of treating myopia, for example, by employing low doses of a muscarinic antagonist, such as atropine, in combination with an adenosine antagonist, such as caffeine.

### Example 2

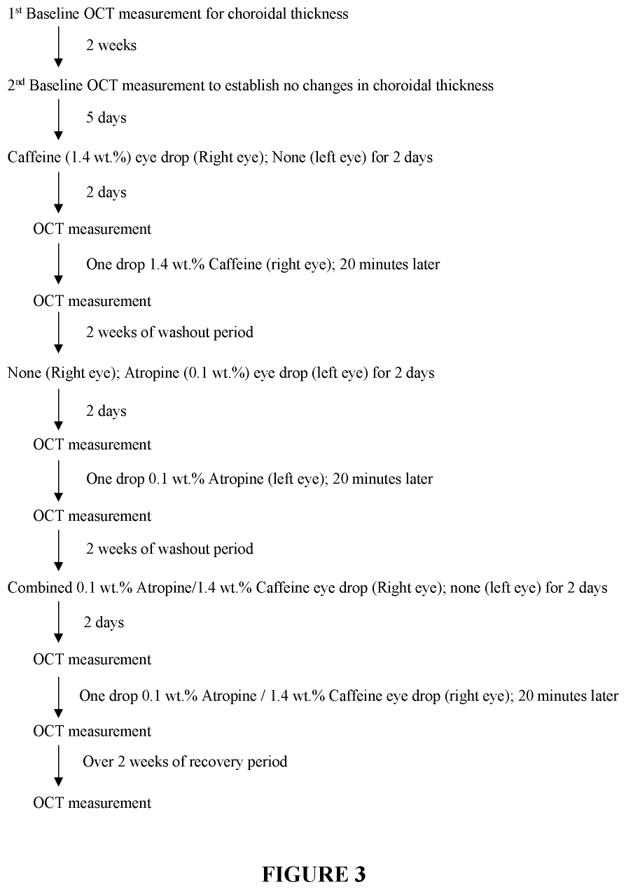

As outlined in FIG. 3, in this experiment, the baseline choroidal thickness of primate 659 was measured via OCT in both eyes at baseline 1 (B1) and was repeated 2 weeks later (B2). Beginning on Day 1 (five days after the second baseline measurement), a single drop of 1.4 wt. % caffeine monotherapy was instilled in the right eye only once per day for 2 days. On Day 3, a choroidal thickness measurement via OCT was then conducted for both eyes and another drop of 1.4 wt. % caffeine monotherapy was instilled in the right eye only and the choroidal thickness measured again in both eyes. Following a washout period of 2 weeks (i.e., beginning on Day 18), a single drop of 0.1 wt. % atropine monotherapy was instilled in the left eye only once per day for 2 days. On Day 20, the choroidal thickness of both eyes was measured using OCT, another drop of 0.1 wt. % atropine monotherapy was instilled in the left eye only, and the choroidal thickness was then measured again in both eyes 20 minutes later. This was then followed by a second washout period for 2 weeks, and beginning on Day 35, 0.1 wt. % atropine/1.4 wt. % caffeine combination therapy was instilled in the right eye only once per day for 2 days. On Day 37, the choroidal thickness of both eyes was measured via OCT, another drop of the 0.1 wt. % atropine/1.4 wt. % caffeine combination therapy was instilled in the right eye only, and the choroidal thickness was then measured again in both eyes 20 minutes later. Following the final measurement on Day 37, no drops were instilled for over 2 weeks for a Recovery Period, after which the choroidal thickness of both eyes was again measured on Day 57. The sequence of events outlined in the procedure illustrated in FIG. 3 is also detailed in Table 3.

TABLE 3

| Day | Left Eye | Right Eye |
|---|---|---|
| Baseline | OCT measured | OCT measured |
| 1 (B1) | | |
| Baseline | OCT measured | OCT measured |
| 2 (B2) | | |
| 1 | No drug | 1.4 wt. % caffeine monotherapy |
| 2 | No drug | 1.4 wt. % caffeine monotherapy |
| 3 | OCT measured, | OCT measured, then 1.4 wt. % |
| | then no drug; | caffeine monotherapy; |
| | OCT measured | OCT measured 20 min. later |
| | 20 min. later | |
| 4-17 | No drug | No drug |
| (No drug) | | |
| 18 | 0.1 wt. % atropine | No drug |
| | monotherapy | |
| 19 | 0.1 wt. % atropine | No drug |
| | monotherapy | |
| 20 | OCT measured, | OCT measured, then no drug; |
| | then 0.1 wt. % | atropine monotherapy; |
| | OCT measured | OCT measured 20 min. later |
| | 20 min. later | |
| 21-34 | No drug | No drug |
| (No drug) | | |
| 35 | No drug | 0.1 wt. % atropine/1.4 wt. % |
| | | caffeine combination therapy |
| 36 | No drug | 0.1 wt. % atropine/1.4 wt. % |
| | | caffeine combination therapy |
| 37 | OCT measured, | OCT measured, then 0.1 wt. % |
| | then no drug; | atropine/1.4 wt. % caffeine |
| | OCT measured | combination therapy; |
| | 20 min. later | OCT measured 20 min. later |
| 38-56 | No drug | No drug |
| (Recovery | | |
| Period) | | |
| 57 | OCT measured | OCT measured |

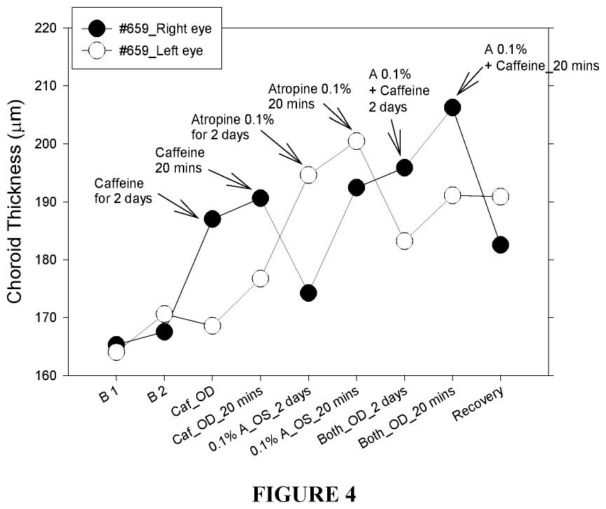

The choroidal thickness results measured during this experiment are shown in FIG. 4; wherein OS refers to left eye (shown as empty circles), and OD refers to right eye (shown as filled-in circles). Choroidal thickness measurements at baseline 1 and 2 of both eyes ranged approximately from about 175 μm to about 180 μm, and was similar between right and left eyes. Use of 0.1 wt. % atropine monotherapy, once per day for 2 days in the right eye only, resulted in an increase in choroidal thickness (increased to about 195 to 200 μm), and similarly use of 1.4 wt. % caffeine monotherapy in the left eye only, once per day for 2 days, resulted in an increase in choroidal thickness. Discontinuation of the use of the 0.1 wt. % atropine monotherapy or the 1.4 wt. % caffeine monotherapy resulted in a decrease in choroidal thickness. Use of a drop of the 0.1 wt. % atropine/1.4 wt. % caffeine combination therapy, once per day for 2 days in the right eye only, also increased the choroidal thickness. The increase in choroidal thickness observed 20 minutes after instillation of a single drop of the 0.1 wt. % atropine/1.4 wt. % caffeine combination therapy was greater than the increase in choroidal thickness observed 20 minutes after the instillation of a single drop of either the 0.1 wt. % atropine monotherapy or the 1.4 wt. % caffeine monotherapy, and significantly, was also greater than additive increase in choroidal thickness observed 20 minutes after the instillation of a single drop of the 0.1 wt. % atropine monotherapy combined with the 1.4 wt. % caffeine monotherapy. The choroidal thickness results measured during this experiment that are shown in FIG. 4, are also provided in Table 4.

TABLE 4

| | Approximate Choroidal | |
|---|---|---|
| | Thickness (μm) | |
| Day | Left Eye | Right Eye |
| Baseline 1 (B1) | 164 | 165 |
| Baseline 2 (B2) | 170 | 167 |
| 3 | 168, 176 | 187, 190 |
| 20 | 194, 200 | 174, 192 |
| 37 | 182, 190 | 195, 207 |
| 57 | 189 | 181 |

This data suggests that administering a combination of a muscarinic antagonist, such as atropine, in combination with an adenosine antagonist, such as caffeine, provides a synergistic effect of increasing choroidal thickness as determined by OCT, relative to the increase in choroidal thickness achieved by either monotherapy. Although adverse effects result from the use of atropine at 0.1 wt. % concentration (data not shown here), this data shows that addition of an adenosine antagonist, such as caffeine, does not diminish the activity of a muscarinic antagonist, such as atropine, and that the addition of the adenosine antagonist may improve upon the efficacy of the muscarinic antagonist. Moreover, this data also suggests that combining an adenosine antagonist with a muscarinic antagonist may provide a pathway to mitigate the adverse side effects associated with muscarinic antagonist monotherapy, such that the adverse side effects may be minimized or reduced, while simultaneously increasing or maintaining the effectiveness of treating myopia, for example, by employing low doses of a muscarinic antagonist, such as atropine, in combination with an adenosine antagonist, such as caffeine.

### Example 3

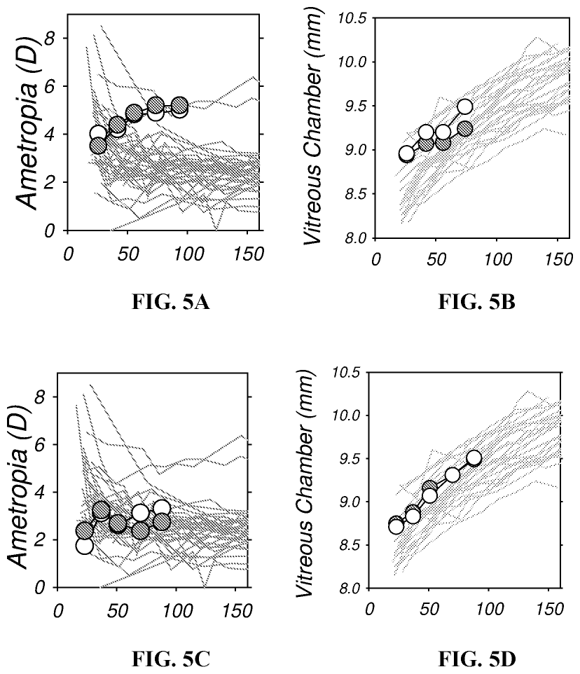

FIGS. 5A-5B provide the details for primate 736 commenced on a −3.00D lens in the right eye (filled circles in FIGS. 5A and 5B) and plano lens in the left (empty circles in FIGS. 5A and 5B) at Day 26. The primate was then dosed with a single eye drop of a 0.02 wt. % atropine composition in both eyes every day until day 94. Baseline spherical equivalent (S.E.) refractive error and axial length in the right and left eye was +3.50D, 8.94 mm and +4.00D, 8.96 mm respectively. Evidence from prior experiments indicates that with the use of −3.00D lens the eye becomes myopic. As seen from FIG. 5A, both eyes continued to increase in hyperopia and the right eye did not develop myopia. On day 94, the spherical equivalent refractive error was +5.19D and +5.00D in the right and left eye, respectively (FIG. 5A), and the vitreous chamber depth was 9.24 mm and 9.49 mm in the right and left eye, respectively (FIG. 5B). The refractive error and vitreous chamber depth data for the right and left eye which are presented in FIGS. 5A-5B, along with the corneal curvature, axial length, lens thickness, and anterior chamber depth data for the right and left eye, are provided in Table 5 (OD=right eye, OS=left eye, OU=both eyes, IOD=intraocular difference). The presented in Table 5 and FIGS. 5A-5B suggests that use of atropine prevents lens induced myopia.

FIGS. 5C-5D provide the details for primate 738 fitted with −3.00D in the right eye (filled circles in FIGS. 5C and 5D) and plano in the left eye (filled circles in FIGS. 5C and 5D) on Day 23 from birth. Both eyes were then dosed with a single eye drop of a combination composition (0.02 wt. % Atropine with 1.4 wt. % caffeine) once every day until Day 88. As seen from FIG. 5C, the refractive error of the eye remained relatively stable throughout the treatment period (Baseline spherical equivalent (S.E.) refractive error in right and left eye was +2.38D and +1.75D, respectively; Day 88 spherical equivalent refractive error was +2.75D and +3.31D, respectively). The vitreous chamber depth on Day 88 was 9.49 mm and 9.51 mm, respectively (Baseline vitreous chamber depth 8.75 mm and 8.71 mm in the right and left eye respectively, as shown in FIG. 5D). The refractive error and vitreous chamber depth data for the right and left eye which are presented in FIGS. 5C-5D, along with the corneal curvature, axial length, lens thickness, and anterior chamber depth data for the right and left eye, are provided in Table 6 (OD=right eye, OS=left eye, OU=both eyes, IOD=intraocular difference). The data presented in Table 6 and FIGS. 5C-5D of this short term evaluation, relative to the data presented in Table 5 and FIGS. 5A-5B, suggests that the efficacy of atropine, for example low concentrations of atropine, such as concentrations of less than 0.05 wt. % atropine, is maintained while combined with caffeine in preventing lens induced myopia.

TABLE 5

| | Refractive Error | | | Corneal Curvature | | | Axial Length | | | Vitrous Chamber | | | Lens Thickness | | | Anterior Chamber | | |
|---|---|---|---|---|---|---|---|---|---|---|---|---|---|---|---|---|---|---|
| Age | (SE.) (D) | | | (SE.) (mm) | | | (mm) | | | Depth (mm) | | | (mm) | | | Depth (mm) | | |
| (days) | OD | OS | IOD | OD | OS | IOD | OD | OS | IOD | OD | OS | IOD | OD | OS | IOD | OD | OS | IOD |
| 26 | 3.50 | 4.00 | −0.50 | 58.08 | 58.46 | −0.37 | 15.40 | 15.43 | −0.03 | 8.94 | 8.96 | −0.02 | 3.82 | 3.76 | 0.06 | 2.64 | 2.71 | −0.07 |
| 42 | 4.38 | 4.19 | 0.19 | 57.60 | 57.27 | 0.33 | 15.59 | 15.64 | −0.05 | 9.07 | 9.20 | −0.13 | 3.69 | 3.82 | −0.13 | 2.83 | 2.73 | 0.10 |
| 56 | 4.88 | 4.81 | 0.06 | 56.23 | 57.00 | −0.78 | 15.67 | 15.78 | −0.11 | 9.08 | 9.20 | −0.12 | 3.81 | 3.73 | 0.08 | 2.79 | 2.84 | −0.05 |
| 74 | 5.19 | 4.88 | 0.31 | 55.31 | 56.21 | −0.89 | 15.85 | 16.17 | −0.32 | 9.24 | 9.49 | −0.25 | 3.78 | 3.79 | −0.01 | 2.83 | 2.89 | −0.06 |
| 94 | 5.19 | 5.00 | 0.19 | — | — | — | — | — | — | — | — | — | — | — | — | — | — | — |

Table Note:
OD: −3D, OS: Plano; OU: 0.02 wt. % Atropine (once daily).

TABLE 6

| | Refractive Error | | | Corneal Curvature | | | Axial Length | | | Vitrous Chamber | | | Lens Thickness | | | Anterior Chamber | | |
|---|---|---|---|---|---|---|---|---|---|---|---|---|---|---|---|---|---|---|
| Age | (SE.) (D) | | | (SE.) (mm) | | | (mm) | | | Depth (mm) | | | (mm) | | | Depth (mm) | | |
| (days) | OD | OS | IOD | OD | OS | IOD | OD | OS | IOD | OD | OS | IOD | OD | OS | IOD | OD | OS | IOD |
| 26 | 2.38 | 1.75 | 0.63 | 60.25 | 60.17 | 0.07 | 14.90 | 14.92 | −0.02 | 8.75 | 8.71 | 0.04 | 3.70 | 3.73 | −0.03 | 2.45 | 2.48 | −0.03 |
| 42 | 3.25 | 3.13 | 0.13 | 59.83 | 60.10 | −0.27 | 15.21 | 15.13 | 0.08 | 8.88 | 8.83 | 0.05 | 3.73 | 3.75 | −0.02 | 2.61 | 2.55 | 0.06 |
| 56 | 2.69 | 2.63 | 0.06 | 58.46 | 58.06 | 0.39 | 15.56 | 15.45 | 0.11 | 9.16 | 9.07 | 0.09 | 3.71 | 3.73 | −0.02 | 2.68 | 2.66 | 0.02 |
| 74 | 2.38 | 3.13 | −0.75 | 58.08 | 56.87 | 1.20 | 15.82 | 15.83 | −0.01 | 9.31 | 9.31 | 0.00 | 3.80 | 3.72 | 0.08 | 2.71 | 2.79 | −0.08 |
| 94 | 2.75 | 3.31 | −0.56 | — | — | — | — | — | — | 9.49 | 9.51 | −0.02 | — | — | — | — | — | — |

Table Note:
OD: −3D, OS: Plano; OU: 0.02 wt. % Atropine with 1.4 wt. % caffeine (once daily).

### Example 4

In a double-blind, cross-over clinical safety assessment study involving 20 human participants, the ocular response to short term use of a single eye drop composition comprising 0.02% atropine with 1.4% caffeine was evaluated. 0.3% hydroxyl-propyl methyl cellulose served as a control. Following a baseline visit, participants were assigned to use either the test eye drop once daily for 5 days or the control eye drop once daily for 5 days following which there was a follow-up visit. At the end of the visit, the use of eye drop was discontinued and followed by a wash out period of 2 nights during which no eye drop was used. Following the wash-out period the remaining eye drop was used once a day for further 5 days. The results of this study, presented in Table 7, suggests all the eye drops did not induce one or more adverse effects, such as any redness (bulbar conjunctival redness or palpebral conjunctival redness), corneal staining, or raised intraocular pressure, for the duration of the study.

TABLE 7

| | | | | | | |
|---|---|---|---|---|---|---|
| Visit | Composition | No. of eyes | Mean | S.D | Min | Max |

Bulbar Conjunctival Redness (Grade 0-4)*

| | | | | | | |
|---|---|---|---|---|---|---|
| Baseline | 1.4% Caffeine + 0.02% Atropine | 40 | 1.96 | 0.31 | 1.50 | 2.50 |
| | 0.3% HPMC | 38 | 2.08 | 0.38 | 1.50 | 3.00 |
| Follow-up | 1.4% Caffeine + 0.02% Atropine | 38 | 2.03 | 0.38 | 1.50 | 2.50 |
| | 0.3% HPMC | 38 | 2.01 | 0.36 | 1.50 | 2.50 |

Palpebral Conjunctival Redness (Grade 04)*

| | | | | | | |
|---|---|---|---|---|---|---|
| Pre Instillation | 1.4% Caffeine + 0.02% Atropine | 40 | 1.73 | 0.37 | 1.00 | 2.50 |
| Visit | 0.3% HPMC | 38 | 1.79 | 0.44 | 1.00 | 3.00 |
| Assessment Visit | 1.4% Caffeine + 0.02% Atropine | 38 | 1.74 | 0.30 | 1.50 | 2.50 |
| | 0.3% HPMC | 38 | 1.64 | 0.35 | 1.00 | 2.50 |

Extent of Overall corneal staining (Grade 04)*

| | | | | | | |
|---|---|---|---|---|---|---|
| Pre Instillation | 1.4% Caffeine + 0.02% Atropine | 40 | 0.30 | 0.42 | 0.00 | 1.50 |
| Visit | 0.3% HPMC | 38 | 0.25 | 0.53 | 0.00 | 2.00 |
| Assessment Visit | 1.4% Caffeine + 0.02% Atropine | 38 | 0.18 | 0.44 | 0.00 | 2.00 |
| | 0.3% HPMC | 38 | 0.30 | 0.54 | 0.00 | 2.00 |

Intraocular Pressure (mm Hg)

| | | | | | | |
|---|---|---|---|---|---|---|
| Pre Instillation | 1.4% Caffeine + 0.02% Atropine | 34 | 12.9 | 2.8 | 8.0 | 18.5 |
| Visit | 0.3% HPMC | 32 | 12.6 | 2.3 | 8.5 | 18.0 |
| Assessment Visit | 1.4% Caffeine + 0.02% Atropine | 34 | 12.8 | 2.7 | 8.5 | 18.0 |
| | 0.3% HPMC | 32 | 12.8 | 2.4 | 8.0 | 18.0 |

Table Note:
Grade 0-4 where 0-none, 1-trace, 2-mild, 3-moderate and 4-severe.

As can be seen from Table 7, redness of the bulbar and palpebral conjunctiva was mild at baseline and did not an increase at the assessment visit conducted approximately 5 days following the baseline visit. An increase in redness of 0.5 grade is considered to berelevant and no such change was seen with the use of the combination (1.4% caffeine with 0.02% atropine) eye drop. Corneal staining was minimal and did not show an increase with the use of the eye drop. Intraocular pressure was within normal limits at baseline and the assessment visit. This study at least demonstrates the safety (mitigation, reduction, and/or avoidance, of adverse side effects associated with muscarinic antagonist monotherapy, such as atropine monotherapy) of the pharmaceutical compositions disclosed herein, such as an eye drop composition, in human participants.

### EXEMPLARY EMBODIMENTS

In an embodiment, a pharmaceutical composition, comprises a muscarinic receptor antagonist and an adenosine receptor antagonist.

In an embodiment, a pharmaceutical composition, comprises a non-selective muscarinic receptor antagonist at a low concentration and an adenosine receptor antagonist.

In an embodiment, a pharmaceutical composition, comprises a non-selective muscarinic receptor antagonist at a concentration of less than 0.05 wt. % and an adenosine receptor antagonist.

In an embodiment, a pharmaceutical composition, comprising: i) a non-selective muscarinic receptor antagonist at a concentration of less than 0.05 wt. %; and ii) a non-selective adenosine receptor antagonist at a concentration from between about 1 to 5 wt %; wherein said pharmaceutical composition when applied to an eye of a subject does not increase the photopic pupil size of the eye beyond about 2 mm.

In an embodiment, an ophthalmic device contains a pharmaceutical composition comprising a muscarinic receptor antagonist and an adenosine receptor antagonist, wherein the ophthalmic device delivers the pharmaceutical composition in a sustained release manner.

In an embodiment, a method of treating myopia in a subject, comprises administering a pharmaceutical composition comprising a muscarinic receptor antagonist and an adenosine receptor antagonist.

In an embodiment, a method of treating myopia in a subject, comprises administering a pharmaceutical composition comprising a muscarinic receptor antagonist and administering a pharmaceutical composition comprising an adenosine receptor antagonist.

In an embodiment, a method of treating myopia in a subject, comprises administering an ophthalmic device containing a pharmaceutical composition comprising a muscarinic receptor antagonist and an adenosine receptor antagonist.

In certain embodiments, one or more than one (including for instance all) of the following further embodiments may comprise each of the other embodiments or parts thereof.

In a further embodiment, the pharmaceutical composition, the ophthalmic device, or the method of treating, of any one of the above embodiments and any one or more of the further embodiments herein, wherein the subject is a human patient.

In a further embodiment, the pharmaceutical composition, the ophthalmic device, or the method of treating, of any one of the above embodiments and any one or more of the further embodiments herein, wherein the subject is a patient in need thereof.

In a further embodiment, the pharmaceutical composition, the ophthalmic device, or the method of treating, of any one of the above embodiments and any one or more of the further embodiments herein, wherein the muscarinic receptor antagonist is a nonselective muscarinic acetylcholinergic antagonist.

In a further embodiment, the pharmaceutical composition, the ophthalmic device, or the method of treating, of any one of the above embodiments and any one or more of the further embodiments herein, wherein the muscarinic receptor antagonist is an M1 selective antagonist.

In a further embodiment, the pharmaceutical composition, the ophthalmic device, or the method of treating, of any one of the above embodiments and any one or more of the further embodiments herein, wherein the muscarinic receptor antagonist is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolomine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, or a pharmaceutically acceptable salt thereof.

In a further embodiment, the pharmaceutical composition, the ophthalmic device, or the method of treating, of any one of the above embodiments and any one or more of the further embodiments herein, wherein the muscarinic receptor antagonist is atropine, or a pharmaceutically acceptable salt thereof.

In a further embodiment, the pharmaceutical composition, the ophthalmic device, or the method of treating, of any one of the above embodiments and any one or more of the further embodiments herein, wherein the muscarinic receptor antagonist is tropine, or a pharmaceutically acceptable salt thereof.

In a further embodiment, the pharmaceutical composition, the ophthalmic device, or the method of treating, of any one of the above embodiments and any one or more of the further embodiments herein, wherein the muscarinic receptor antagonist is tropic acid.

In a further embodiment, the pharmaceutical composition, the ophthalmic device, or the method of treating, of any one of the above embodiments and any one or more of the further embodiments herein, wherein the muscarinic receptor antagonist is present in an amount in of less than 0.05 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is present in an amount in the range from between approximately 0.001 wt. % to less than 0.05 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is present in an amount in the range from between approximately 0.001 wt. % to less than 0.05 wt. %, such as between approximately 0.001-0.045 wt. %, between approximately 0.001-0.04 wt. %, between approximately 0.001-0.035 wt. %, between approximately 0.001-0.03 wt. %, between approximately 0.001-0.025 wt. %, between approximately 0.001-0.02 wt. %, between approximately 0.001-0.015 wt. %, between approximately 0.001-0.01 wt. %, between approximately 0.001-0.005 wt. %, between approximately 0.005-0.03 wt. %, between approximately 0.005-0.04 wt. %, between approximately 0.01-0.03 wt. %, between approximately 0.01-0.045 wt. %, between approximately 0.01-0.04 wt. %, between approximately 0.02-0.04 wt. %, between approximately 0.02-0.03 wt. %, between approximately 0.015-0.025 wt. %, between approximately 0.015-0.03 wt. %, or between approximately 0.015-0.035 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is present in an amount of approximately 0.001 wt. %, approximately 0.002 wt. %, approximately 0.005 wt. %, approximately 0.01 wt. %, approximately 0.015 wt. %, approximately 0.02 wt. %, approximately 0.025 wt. %, approximately 0.03 wt. %, approximately 0.035 wt. %, approximately 0.04 wt. %, or approximately 0.045 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is used in low concentrations, such as used in concentrations of less than 0.05 wt. %, for example, used in concentrations of between less than about 0.05 wt. % to no less than 0.001 wt. %, such as in concentrations of approximately 0.045 wt. % or less, approximately 0.04 wt. % or less, approximately 0.035 wt. % or less, or approximately 0.03 wt. % or less, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is used in low concentrations, such as used in concentrations of less than 0.05 wt. %, for example, used in concentrations of between less than about 0.05 wt. % to no less than 0.001 wt. %, such as in concentrations of approximately 0.045 wt. % or less, approximately 0.04 wt. % or less, approximately 0.035 wt. % or less, or approximately 0.03 wt. % or less, or in the range of between less than 0.05 wt. % to 0.001 wt. %, such as, between approximately 0.045 wt. % to 0.001 wt. %, between approximately 0.04 wt. % to 0.001 wt. %, between approximately 0.035 wt. % to 0.001 wt. %, between approximately 0.03 wt. % to 0.001 wt. %, between approximately 0.025 wt. % to 0.001 wt. %, between approximately 0.02 wt. % to 0.001 wt. %, between approximately 0.015 wt. % to 0.001 wt. %, between approximately 0.01 wt. % to 0.001 wt. %, between <0.01 wt. % to 0.001 wt. %, between approximately 0.045 wt. % to 0.01 wt. %, between approximately 0.04 wt. % to 0.02 wt. %, between approximately 0.03 wt. % to 0.02 wt. %, or between approximately 0.03 wt. % to 0.01 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.001 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.005 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.01 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.02 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.03 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present in a concentration of approximately 0.04 wt. %, relative to the pharmaceutical composition.

In a further embodiment, the pharmaceutical composition, the ophthalmic device, or the method of treating, of any one of the above embodiments and any one or more of the further embodiments herein, wherein the adenosine receptor antagonist is a non-selective adenosine receptor antagonist.

In a further embodiment, the pharmaceutical composition, the ophthalmic device, or the method of treating, of any one of the above embodiments and any one or more of the further embodiments herein, wherein the non-selective adenosine receptor antagonist is a xanthine derivative, or a pharmaceutically acceptable salt thereof.

In a further embodiment, the pharmaceutical composition, the ophthalmic device, or the method of treating, of any one of the above embodiments and any one or more of the further embodiments herein, wherein the non-selective adenosine receptor antagonist is caffeine, or a pharmaceutically acceptable salt thereof.

In a further embodiment, the pharmaceutical composition, the ophthalmic device, or the method of treating, of any one of the above embodiments and any one or more of the further embodiments herein, wherein the non-selective adenosine receptor antagonist is caffeine citrate.

In a further embodiment, the pharmaceutical composition, the ophthalmic device, or the method of treating, of any one of the above embodiments and any one or more of the further embodiments herein, wherein the non-selective adenosine receptor antagonist is 7-methylxanthine, or a pharmaceutically acceptable salt thereof.

In a further embodiment, the pharmaceutical composition, the ophthalmic device, or the method of treating, of any one of the above embodiments and any one or more of the further embodiments herein, wherein the adenosine receptor antagonist is present in an amount in the range of between approximately 0.1-5.0 wt. %, relative to the pharmaceutical composition.

In a further embodiment, the pharmaceutical composition, the ophthalmic device, or the method of treating, of any one of the above embodiments and any one or more of the further embodiments herein, wherein the adenosine receptor antagonist is present in an amount in the range of between approximately 0.1-4.0 wt. %, between approximately 0.1-3.0 wt. %, between approximately 0.1-2.0 wt. %, between approximately 0.1-1.0 wt. %, between approximately 0.5-5.0 wt. %, between approximately 1.0-5.0 wt. %, between 1.0-2.0 wt. %, between approximately 2.0-5.0 wt. %, between approximately 3.0-5.0 wt. %, or between approximately 4.0-5.0 wt. %, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present at a concentration in the range of approximately 0.01-0.04%, an adenosine receptor antagonist is caffeine and is present at a concentration in the range of approximately 0.5-3.0%, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present at a concentration in the range of approximately 0.02-0.04%, an adenosine receptor antagonist is caffeine and is present at a concentration in the range of approximately 1.0-2.0%, relative to the pharmaceutical composition.

In a further embodiment, the pharmaceutical composition, the ophthalmic device, or the method of treating, of any one of the above embodiments and any one or more of the further embodiments herein, wherein the muscarinic receptor antagonist is atropine and is present at a concentration of approximately 0.03%, an adenosine receptor antagonist is caffeine and is present at a concentration in the range of approximately 0.5-3.0%, relative to the pharmaceutical composition.

In a further embodiment, the pharmaceutical composition, the ophthalmic device, or the method of treating, of any one of the above embodiments and any one or more of the further embodiments herein, wherein the pharmaceutical composition is an aqueous composition.

In a further embodiment, the pharmaceutical composition, the ophthalmic device, or the method of treating, of any one of the above embodiments and any one or more of the further embodiments herein, wherein the pharmaceutical composition is an ophthalmic formulation.

In a further embodiment, the pharmaceutical composition, the ophthalmic device, or the method of treating, of any one of the above embodiments and any one or more of the further embodiments herein, wherein the pharmaceutical composition is an ophthalmic aqueous formulation.

In a further embodiment, the pharmaceutical composition, the ophthalmic device, or the method of treating, of any one of the above embodiments and any one or more of the further embodiments herein, wherein the pharmaceutical composition is an eye drop formulation.

In a further embodiment, the pharmaceutical composition, the ophthalmic device, or the method of treating, of any one of the above embodiments and any one or more of the further embodiments herein, wherein the pharmaceutical composition is an ocular spray formulation.

In a further embodiment, the pharmaceutical composition, the ophthalmic device, or the method of treating, of any one of the above embodiments and any one or more of the further embodiments herein, wherein the pharmaceutical composition is an ocular pharmaceutical composition contained within a contact lens blister pack.

In a further embodiment, the pharmaceutical composition, the ophthalmic device, or the method of treating, of any one of the above embodiments and any one or more of the further embodiments herein, wherein the pharmaceutical composition is a topical formulation.

In a further embodiment, the pharmaceutical composition, the ophthalmic device, or the method of treating, of any one of the above embodiments and any one or more of the further embodiments herein, wherein the pharmaceutical composition is an ophthalmic composition.

In a further embodiment, the pharmaceutical composition, the ophthalmic device, or the method of treating, of any one of the above embodiments and any one or more of the further embodiments herein, wherein the pharmaceutical composition is a topical ophthalmic composition.

In a further embodiment, the pharmaceutical composition, the ophthalmic device, or the method of treating, of any one of the above embodiments and any one or more of the further embodiments herein, wherein the ophthalmic composition is a topical ophthalmic composition.

In a further embodiment, the pharmaceutical composition, the ophthalmic device, or the method of treating, of any one of the above embodiments and any one or more of the further embodiments herein, wherein the pharmaceutical composition is an ocular gel formulation.

In a further embodiment, the pharmaceutical composition, the ophthalmic device, or the method of treating, of any one of the above embodiments and any one or more of the further embodiments herein, wherein the pharmaceutical composition is an ophthalmic emulsion.

In a further embodiment, the pharmaceutical composition, the ophthalmic device, or the method of treating, of any one of the above embodiments and any one or more of the further embodiments herein, wherein the pharmaceutical composition is ophthalmic liposomes.

In a further embodiment, the pharmaceutical composition, the ophthalmic device, or the method of treating, of any one of the above embodiments and any one or more of the further embodiments herein, wherein the pharmaceutical composition is nano wafers.

In a further embodiment, the pharmaceutical composition, the ophthalmic device, or the method of treating, of any one of the above embodiments and any one or more of the further embodiments herein, wherein the pharmaceutical composition is a nano particle suspension.

In a further embodiment, the pharmaceutical composition, the ophthalmic device, or the method of treating, of any one of the above embodiments and any one or more of the further embodiments herein, wherein the pharmaceutical composition is an ophthalmic ointment.

In a further embodiment, the pharmaceutical composition, the ophthalmic device, or the method of treating, of any one of the above embodiments and any one or more of the further embodiments herein, wherein the pharmaceutical composition further comprises one or more additional ophthalmically acceptable excipients and additives, comprising carriers, stabilizers, an osmolarity adjusting agent, a preservative, a buffer agent, a tonicity adjusting agent, thickeners, or other excipients.

In a further embodiment, the pharmaceutical composition, the ophthalmic device, or the method of treating, of any one of the above embodiments and any one or more of the further embodiments herein, wherein the carriers is selected from the group consisting of: water, mixture of water and water-miscible solvent, vegetable or mineral oils comprising from 0.1 to 5% by weight hydroxyethylcellulose, ethyl oleate, carboxymethylocelluse, carboxy methycellulose, hydroxymethylcellolose, hydroxyethylcellulose, ethyl acrulate, polyacrylamide, pectin, alginates, starch derivatives, polyvinyl alcohol, polyvinylpyrrolidone, polyvinyl methyl ether, polyethylene oxide, cross-linked polyacrylic acid, Carbopol, lecithin, polyoxyethylene stearate, heptadecaethyleneoxycetanol, or polyoxyethylene sorbitol monooleate.

In a further embodiment, the pharmaceutical composition, the ophthalmic device, or the method of treating, of any one of the above embodiments and any one or more of the further embodiments herein, wherein the osmolarity adjusting agent is sodium chloride.

In a further embodiment, the pharmaceutical composition, the ophthalmic device, or the method of treating, of any one of the above embodiments and any one or more of the further embodiments herein, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

In a further embodiment, the pharmaceutical composition, the ophthalmic device, or the method of treating, of any one of the above embodiments and any one or more of the further embodiments herein, wherein the buffer agent is selected from borates, borate-polyol complexes, phosphate buffering agents, citrate buffering agents, acetate buffering agents, carbonate buffering agents, organic buffering agents, amino acid buffering agents, or combinations thereof.

In a further embodiment, the pharmaceutical composition, the ophthalmic device, or the method of treating, of any one of the above embodiments and any one or more of the further embodiments herein, wherein the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In a further embodiment, the pharmaceutical composition, the ophthalmic device, or the method of treating, of any one of the above embodiments and any one or more of the further embodiments herein, wherein the pharmaceutical composition is a sustained release formulation or a subconjunctival depot.

In a further embodiment, the pharmaceutical composition, the ophthalmic device, or the method of treating, of any one of the above embodiments and any one or more of the further embodiments herein, wherein the pharmaceutical composition is a sustained release formulation.

In a further embodiment, the pharmaceutical composition, the ophthalmic device, or the method of treating, of any one of the above embodiments and any one or more of the further embodiments herein, wherein the pharmaceutical composition is a sustained release formulation contained within an ophthalmic device.

In a further embodiment, the pharmaceutical composition, the ophthalmic device, or the method of treating, of any one of the above embodiments and any one or more of the further embodiments herein, wherein the pharmaceutical composition is a contained within an ophthalmic device.

In a further embodiment, the pharmaceutical composition, the ophthalmic device, or the method of treating, of any one of the above embodiments and any one or more of the further embodiments herein, wherein the pharmaceutical composition is an ophthalmic composition, and the ophthalmic composition is contained within an ophthalmic device.

In a further embodiment, the pharmaceutical composition, the ophthalmic device, or the method of treating, of any one of the above embodiments and any one or more of the further embodiments herein, wherein the ophthalmic device is a contact lens, an ocular insert, a corneal onlay, a corneal inlay, a nano wafer, a liposome, a nanoparticle, a punctal plug, or a hydrogel matrix with microfluid reservoir.

In a further embodiment, the pharmaceutical composition, the ophthalmic device, or the method of treating, of any one of the above embodiments and any one or more of the further embodiments herein, wherein the ophthalmic device is a contact lens.

In a further embodiment, the pharmaceutical composition, the ophthalmic device, or the method of treating, of any one of the above embodiments and any one or more of the further embodiments herein, wherein the ophthalmic device is an ocular insert.

In a further embodiment, the pharmaceutical composition, the ophthalmic device, or the method of treating, of any one of the above embodiments and any one or more of the further embodiments herein, wherein the ophthalmic device is a corneal onlay.

In a further embodiment, the pharmaceutical composition, the ophthalmic device, or the method of treating, of any one of the above embodiments and any one or more of the further embodiments herein, wherein the ophthalmic device is a corneal inlay.

In a further embodiment, the pharmaceutical composition, the ophthalmic device, or the method of treating, of any one of the above embodiments and any one or more of the further embodiments herein, wherein the ophthalmic device is a nano wafer.

In a further embodiment, the pharmaceutical composition, the ophthalmic device, or the method of treating, of any one of the above embodiments and any one or more of the further embodiments herein, wherein the ophthalmic device is a liposome.

In a further embodiment, the pharmaceutical composition, the ophthalmic device, or the method of treating, of any one of the above embodiments and any one or more of the further embodiments herein, wherein the ophthalmic device is a nanoparticle.

In a further embodiment, the pharmaceutical composition, the ophthalmic device, or the method of treating, of any one of the above embodiments and any one or more of the further embodiments herein, wherein the ophthalmic device is a punctal plug.

In a further embodiment, the pharmaceutical composition, the ophthalmic device, or the method of treating, of any one of the above embodiments and any one or more of the further embodiments herein, wherein the ophthalmic device is a hydrogel matrix with microfluid reservoir.

In a further embodiment, the pharmaceutical composition, the ophthalmic device, or the method of treating, of any one of the above embodiments and any one or more of the further embodiments herein, wherein the ophthalmic device delivers the pharmaceutical composition in a sustained release manner.

In a further embodiment, the pharmaceutical composition, the ophthalmic device, or the method of treating, of any one of the above embodiments and any one or more of the further embodiments herein, wherein the pharmaceutical composition is formulated as an ophthalmic composition for treatment of an ophthalmic disorder or condition.

In a further embodiment, the pharmaceutical composition, the ophthalmic device, or the method of treating, of any one of the above embodiments and any one or more of the further embodiments herein, wherein the pharmaceutical composition is formulated as an ophthalmic composition for treatment of pre-myopia, myopia, or progression of myopia.

In a further embodiment, the pharmaceutical composition, the ophthalmic device, or the method of treating, of any one of the above embodiments and any one or more of the further embodiments herein, wherein the pharmaceutical composition is formulated as an ophthalmic composition for treatment of high myopia, moderate myopia, or low myopia.

In a further embodiment, the pharmaceutical composition, the ophthalmic device, or the method of treating, of any one of the above embodiments and any one or more of the further embodiments herein, wherein the pharmaceutical composition is formulated as an ophthalmic composition for treatment of a patient diagnosed as pre-myopic (or at risk of developing myopia).

In a further embodiment, the pharmaceutical composition, the ophthalmic device, or the method of treating, of any one of the above embodiments and any one or more of the further embodiments herein, wherein the pharmaceutical composition is distributed with substantial uniformity throughout the ophthalmic device.

In a further embodiment, the pharmaceutical composition, the ophthalmic device, or the method of treating, of any one of the above embodiments and any one or more of the further embodiments herein, wherein the ophthalmic device is contained within a contact lens blister pack.

In a further embodiment, the pharmaceutical composition, the ophthalmic device, or the method of treating, of any one of the above embodiments and any one or more of the further embodiments herein, wherein the pharmaceutical composition bathes the ophthalmic device within the contact lens blister pack.

In a further embodiment, the pharmaceutical composition, the ophthalmic device, or the method of treating, of any one of the above embodiments and any one or more of the further embodiments herein, wherein the muscarinic antagonist and the adenosine antagonist are co-administered concurrently, co-administered sequentially with administration of the muscarinic antagonist followed by the adenosine antagonist, or co-administered sequentially with administration of the adenosine antagonist followed by the muscarinic antagonist.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises a muscarinic receptor antagonist conjugated with an adenosine receptor antagonist.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises one or more molecules of a muscarinic receptor antagonist conjugated with one or more molecules of an adenosine receptor antagonist.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises one molecule of atropine conjugated with one molecule of caffeine, such as a conjugate compound of Formula (I), Formula (II) or Formula (III):

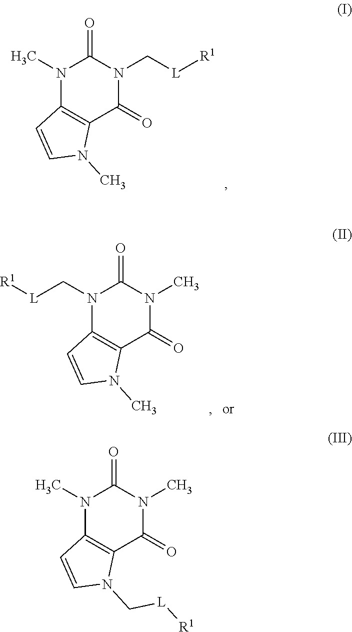

wherein R¹ is an atropine moiety, and wherein L is a divalent linker, such that the divalent linker group covalently conjugates an atropine molecule with a caffeine molecule.

In a further embodiment, the pharmaceutical composition, the ophthalmic device, or the method of treating, of any one of the above embodiments and any one or more of the further embodiments herein, wherein the divalent linker (L) is a hydrocarbon linker comprising stable bonds, such as hydrocarbon linker that is hydrophobic, for example, divalent linker (L) comprises a polyalkyl linker, e.g., C₅-C₂₀ alkyl linker; a C₅-C₆ cycloalkyl linker, e.g., 1,4-cyclohexyl linker, 1,3-cyclohexyl linker, 1,2-cyclohexyl linker, 1,3-cyclopentyl, or 1,2-cyclopentyl; a C₅-C₆ cycloalkenyl linker.

In a further embodiment, the pharmaceutical composition, the ophthalmic device, or the method of treating, of any one of the above embodiments and any one or more of the further embodiments herein, wherein the divalent linker (L) is a divalent linker having stable bonds that is hydrophilic, for example, divalent linker (L) comprises a polyethylene glycol linker, e.g., —(OCH₂CH₂)ₙ—, wherein n is 5-20.

In a further embodiment, the pharmaceutical composition, the ophthalmic device, or the method of treating, of any one of the above embodiments and any one or more of the further embodiments herein, wherein the divalent linker (L) is a divalent linker having ester linkages susceptible to hydrolysis by esterases, for example, divalent linker (L) comprises an acetyl linker, e.g., —(O(CO)CH₂)—.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises one molecule of atropine conjugated to two molecules of caffeine, such as a conjugate compound of Formula (IV):

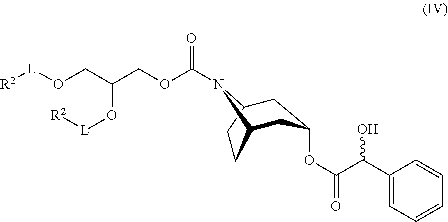

wherein an N-carbamate derivative of atropine is conjugated via a trivalent linker, such as a 1,2,3-propane triol moiety, to two divalent linkers (L), wherein L may independently be a polyethylene glycol linker (wherein n is independently 5-20), a polyalkyl linker, e.g., C₅-C₂₀ alkyl linker: a C₅-C₆ cycloalkyl linker: a C₅-C₆ cycloalkenyl linker; or an ester linkage, such as an acetyl linker, e.g., —(O(CO)CH₂)—, and wherein each of the two independent divalent linkers are further conjugated to R² groups, wherein R² is independently a caffeine moiety independently conjugated via the N-methyl group at the N1, N3, or N7 position.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises two molecules of atropine conjugated to one molecule of caffeine, such as a conjugate compound of Formula (V):

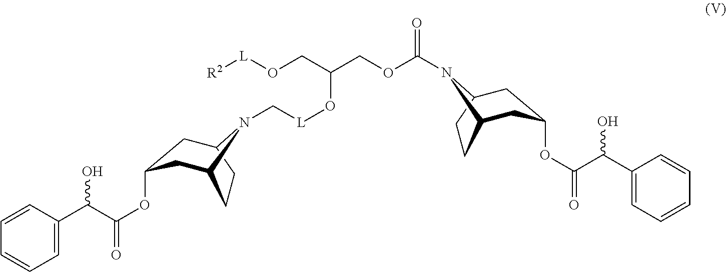

wherein an N-carbamate derivative of atropine is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker, a C₅-C₆ cycloalkyl linker, a C₅-C₆ cycloalkenyl linker; or an ester linkage, such as an acetyl linker, e.g., —(O(CO)CH₂)—, wherein one of the independent divalent linkers are further conjugated to an atropine moiety via the N-methyl group, and wherein one of the independent divalent linkers are further conjugated to an R² group, wherein R² is a caffeine moiety conjugated via the N-methyl group at the N1, N3, or N7 position.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises one molecule of tropine conjugated with one molecule of caffeine, such as a conjugate compound of Formula (VI):

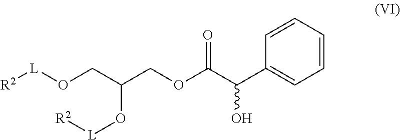

wherein a tropine moiety is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker, a C₅-C₆ cycloalkyl linker, a C₅-C₆ cycloalkenyl linker; or an ester linkage, such as an acetyl linker, e.g., —(O(CO)CH₂)—, and wherein each of the two independent divalent linkers are further conjugated to R² groups, wherein R² is independently a caffeine moiety independently conjugated via the N-methyl group at the N1, N3, or N7 position.

In a further embodiment, the pharmaceutical composition, the ophthalmic device, or the method of treating, of any one of the above embodiments and any one or more of the further embodiments herein, wherein the pharmaceutical composition may comprise, the ophthalmic device may comprise, or the method of treating myopia in a patient in need thereof may comprise administering, a hybrid molecule that comprises two molecules of tropine conjugated to one molecule of caffeine, such as a conjugate compound of Formula (VII):

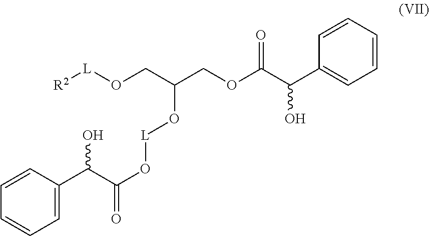

wherein a tropine moiety is conjugated via a trivalent linker, such as a 1,2,3-propane triol moiety, to two independent divalent linkers (L), wherein L may independently be a polyethylene glycol linker (wherein n is independently 5-20), a polyalkyl linker, e.g., C₅-C₂₀ alkyl linker; a C₅-C₆ cycloalkyl linker; a C₅-C₆ cycloalkenyl linker; or an ester linkage, such as an acetyl linker, e.g., —(O(CO)CH₂)—, wherein one of the independent divalent linkers are further conjugated to a tropine moiety, and wherein one of the independent divalent linkers are further conjugated to an R² group, wherein R² is a caffeine moiety conjugated via the N-methyl group at the N1, N3, or N7 position.

In a further embodiment, the pharmaceutical composition, the ophthalmic device, or the method of treating, of any one of the above embodiments and any one or more of the further embodiments herein, wherein the hybrid molecule is a conjugate compound of Formula (I).

In a further embodiment, the pharmaceutical composition, the ophthalmic device, or the method of treating, of any one of the above embodiments and any one or more of the further embodiments herein, wherein the hybrid molecule is a conjugate compound of Formula (II).

In a further embodiment, the pharmaceutical composition, the ophthalmic device, or the method of treating, of any one of the above embodiments and any one or more of the further embodiments herein, wherein the hybrid molecule is a conjugate compound of Formula (III).

In a further embodiment, the pharmaceutical composition, the ophthalmic device, or the method of treating, of any one of the above embodiments and any one or more of the further embodiments herein, wherein the hybrid molecule is a conjugate compound of Formula (IV).

In a further embodiment, the pharmaceutical composition, the ophthalmic device, or the method of treating, of any one of the above embodiments and any one or more of the further embodiments herein, wherein the hybrid molecule is a conjugate compound of Formula (V).

In a further embodiment, the pharmaceutical composition, the ophthalmic device, or the method of treating, of any one of the above embodiments and any one or more of the further embodiments herein, wherein the hybrid molecule is a conjugate compound of Formula (VI).

In a further embodiment, the pharmaceutical composition, the ophthalmic device, or the method of treating, of any one of the above embodiments and any one or more of the further embodiments herein, wherein the hybrid molecule is a conjugate compound of Formula (VII).

In a further embodiment, the pharmaceutical composition, the ophthalmic device, or the method of treating, of any one of the above embodiments and any one or more of the further embodiments herein, wherein the divalent linker (L) independently represents or comprises a polyalkyl linker, e.g., C₅-C₂₀ alkyl linker; a C₅-C₆ cycloalkyl linker, e.g., 1,4-cyclohexyl linker, 1,3-cyclohexyl linker, 1,2-cyclohexyl linker, 1,3-cyclopentyl, or 1,2-cyclopentyl; a C₅-C₆ cycloalkenyl linker.

In a further embodiment, the pharmaceutical composition, the ophthalmic device, or the method of treating, of any one of the above embodiments and any one or more of the further embodiments herein, wherein the divalent linker (L) independently represents or comprises a polyethylene glycol linker, e.g., —(OCH₂CH₂)ₙ—, wherein n is 5-20.

In a further embodiment, the pharmaceutical composition, the ophthalmic device, or the method of treating, of any one of the above embodiments and any one or more of the further embodiments herein, wherein the divalent linker (L) independently represents or comprises an acetyl linker. e.g., —(O(CO)CH₂)—.

In a further embodiment, the pharmaceutical composition, the ophthalmic device, or the method of treating, of any one of the above embodiments and any one or more of the further embodiments herein, wherein the method prevents the progression of myopia in the treated patient.

In a further embodiment, the pharmaceutical composition, the ophthalmic device, or the method of treating, of any one of the above embodiments and any one or more of the further embodiments herein, wherein the method controls the progression of myopia in the treated patient.

In a further embodiment, the pharmaceutical composition, the ophthalmic device, or the method of treating, of any one of the above embodiments and any one or more of the further embodiments herein, wherein the method slows, reduces, retards, and/or mitigates the progression of myopia in the treated patient.

In a further embodiment, the pharmaceutical composition, the ophthalmic device, or the method of treating, of any one of the above embodiments and any one or more of the further embodiments herein, wherein the method controls, slows, reduces, retards, and/or mitigates the progression of myopia in the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between approximately 50-90%, or between approximately 75-90%, relative to non-treatment.

In a further embodiment, the pharmaceutical composition, the ophthalmic device, or the method of treating, of any one of the above embodiments and any one or more of the further embodiments herein, wherein the use of the pharmaceutical composition, the ophthalmic device or the method of treating limits the increase in photopic pupil size of the eye of the user to about 1-2 mm, about 1 mm, about 2 mm, less than 2 mm, less than 1 mm.

In a further embodiment, the pharmaceutical composition, the ophthalmic device, or the method of treating, of any one of the above embodiments and any one or more of the further embodiments herein, wherein the use of the pharmaceutical composition, the ophthalmic device or the method of treating does not increase the photopic pupil size of the eye beyond about 2 mm.

In a further embodiment, the pharmaceutical composition, the ophthalmic device, or the method of treating, of any one of the above embodiments and any one or more of the further embodiments herein, wherein the use of the pharmaceutical composition, the ophthalmic device or the method of treating limits the reduction in accommodative amplitude of the eye of the user to about 1.0-6.0D, 1.0-5.0D, 1.0-4.0D, 1.0-3.0D, 1.0-2.0D, less than 6.0D, less than 5.0D, less than 4.0D, less than 3.0D, less than 2.0D and less than 1.0D.

In a further embodiment, the pharmaceutical composition, the ophthalmic device, or the method of treating, of any one of the above embodiments and any one or more of the further embodiments herein, wherein the use of the pharmaceutical composition, the ophthalmic device or the method of treating does not decrease the amplitude of accommodation of the eye beyond about 6.0D.

In a further embodiment, the pharmaceutical composition, the ophthalmic device, or the method of treating, of any one of the above embodiments and any one or more of the further embodiments herein, wherein: i) the ophthalmic composition does not increase the photopic pupil size of an eye beyond 2 mm; and/or ii) does not decrease the amplitude of accommodation of the eye beyond about 6.0D.

In a further embodiment, the pharmaceutical composition, the ophthalmic device, or the method of treating, of any one of the above embodiments and any one or more of the further embodiments herein, wherein the ophthalmic composition does not increase the photopic pupil size of an eye beyond 2 mm.

In a further embodiment, the pharmaceutical composition, the ophthalmic device, or the method of treating, of any one of the above embodiments and any one or more of the further embodiments herein, wherein the ophthalmic composition does not decrease the amplitude of accommodation of the eye beyond about 6.0D.

In a further embodiment, the pharmaceutical composition, the ophthalmic device, or the method of treating, of any one of the above embodiments and any one or more of the further embodiments herein, wherein the method prevents or reverses the progression of myopia in the treated patient.

In a further embodiment, the pharmaceutical composition, the ophthalmic device, or the method of treating, of any one of the above embodiments and any one or more of the further embodiments herein, wherein the patient suffers from high myopia.

In a further embodiment, the pharmaceutical composition, the ophthalmic device, or the method of treating, of any one of the above embodiments and any one or more of the further embodiments herein, wherein the patient suffers from moderate myopia.

In a further embodiment, the pharmaceutical composition, the ophthalmic device, or the method of treating, of any one of the above embodiments and any one or more of the further embodiments herein, wherein the patient suffers from low myopia.

In a further embodiment, the pharmaceutical composition, the ophthalmic device, or the method of treating, of any one of the above embodiments and any one or more of the further embodiments herein, wherein the patient is diagnosed as pre-myopic (or at risk of developing myopia).

In a further embodiment, the pharmaceutical composition, the ophthalmic device, or the method of treating, of any one of the above embodiments and any one or more of the further embodiments herein, wherein the method increases choroidal thickness of an eye of the treated patient.

In a further embodiment, the pharmaceutical composition, the ophthalmic device, or the method of treating, of any one of the above embodiments and any one or more of the further embodiments herein, wherein the method increases choroidal thickness of an eye of the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between 5 approximately 0-90%, or between approximately 75-90%, relative to non-treatment.

In a further embodiment, the pharmaceutical composition, the ophthalmic device, or the method of treating, of any one of the above embodiments and any one or more of the further embodiments herein, wherein the method prevents axial (or longitudinal) growth of an eye of the treated patient.

In a further embodiment, the pharmaceutical composition, the ophthalmic device, or the method of treating, of any one of the above embodiments and any one or more of the further embodiments herein, wherein the method controls, slows, reduces, retards, and/or mitigates axial (or longitudinal) growth of an eye of the treated patient.

In a further embodiment, the pharmaceutical composition, the ophthalmic device, or the method of treating, of any one of the above embodiments and any one or more of the further embodiments herein, wherein the method controls, slows, reduces, retards, and/or mitigates axial (or longitudinal) growth of an eye of the treated patient in the range of between 5-100%, relative to non-treatment.

In a further embodiment, the pharmaceutical composition, the ophthalmic device, or the method of treating, of any one of the above embodiments and any one or more of the further embodiments herein, wherein the method controls, slows, reduces, retards, and/or mitigates axial (or longitudinal) growth of an eye of the treated patient in the range of between approximately 5-95%, between approximately 5-90%, between approximately 5-80%, between approximately 5-70%, between approximately 5-60%, between approximately 5-50%, between approximately 5-40%, between approximately 5-30%, between approximately 5-20%, between approximately 10-100%, between approximately 20-90%, between approximately 25-90%, between approximately 30-90%, between approximately 40-90%, between approximately 50-90%, or between approximately 75-90%, relative to non-treatment.

In a further embodiment, the pharmaceutical composition, the ophthalmic device, or the method of treating, of any one of the above embodiments and any one or more of the further embodiments herein, wherein the method: i) increases choroidal thickness of an eye of the treated patient, relative to non-treatment; and/or ii) reduces axial (or longitudinal) growth of an eye of the treated patient, relative to non-treatment.

In a further embodiment, the pharmaceutical composition, the ophthalmic device, or the method of treating, of any one of the above embodiments and any one or more of the further embodiments herein, wherein the patient is treated for a period of between about 1 month to 10 years.

In a further embodiment, the pharmaceutical composition, the ophthalmic device, or the method of treating, of any one of the above embodiments and any one or more of the further embodiments herein, wherein the patient is treated for a period of at least 6 months, at least 1 year, at least 2 years, at least 3 years, at least 5 years, at least 7 years, or at least 9 years.

In a further embodiment, the pharmaceutical composition, the ophthalmic device, or the method of treating, of any one of the above embodiments and any one or more of the further embodiments herein, wherein the method results in less severe adverse side effects, relative to atropine monotherapy.

In a further embodiment, the pharmaceutical composition, the ophthalmic device, or the method of treating, of any one of the above embodiments and any one or more of the further embodiments herein, wherein the treated patient suffers from less severe adverse side effects according to the use of the pharmaceutical composition disclosed herein, to the use of the ophthalmic device disclosed herein, or the method of treating disclosed herein, relative to atropine monotherapy.

In a further embodiment, the pharmaceutical composition, the ophthalmic device, or the method of treating, of any one of the above embodiments and any one or more of the further embodiments herein, wherein the method results in a smaller increase of pupil size relative to atropine monotherapy.

In a further embodiment, the pharmaceutical composition, the ophthalmic device, or the method of treating, of any one of the above embodiments and any one or more of the further embodiments herein, wherein the treated patient has a smaller increase of pupil size according to the use of the pharmaceutical composition disclosed herein, to the use of the ophthalmic device disclosed herein, or the method of treating disclosed herein, relative to atropine monotherapy.

In a further embodiment, the pharmaceutical composition, the ophthalmic device, or the method of treating, of any one of the above embodiments and any one or more of the further embodiments herein, wherein the method results in a smaller decrease in accommodative amplitude, relative to atropine monotherapy.

In a further embodiment, the pharmaceutical composition, the ophthalmic device, or the method of treating, of any one of the above embodiments and any one or more of the further embodiments herein, wherein the treated patient has a smaller decrease in accommodative amplitude according to the use of the pharmaceutical composition disclosed herein, to the use of the ophthalmic device disclosed herein, or the method of treating disclosed herein, relative to atropine monotherapy.

In a further embodiment, the pharmaceutical composition, the ophthalmic device, or the method of treating, of any one of the above embodiments and any one or more of the further embodiments herein, wherein the pharmaceutical composition is ophthalmically administered to an eye of the patient.

In a further embodiment, the pharmaceutical composition, the ophthalmic device, or the method of treating, of any one of the above embodiments and any one or more of the further embodiments herein, wherein the pharmaceutical composition is topically administered.

In a further embodiment, the pharmaceutical composition, the ophthalmic device, or the method of treating, of any one of the above embodiments and any one or more of the further embodiments herein, wherein the pharmaceutical composition is administered to the eye in the form of an eye drop formulation, an ocular spray formulation, or an ocular gel formulation.

In a further embodiment, the pharmaceutical composition, the ophthalmic device, or the method of treating, of any one of the above embodiments and any one or more of the further embodiments herein, wherein the pharmaceutical composition is administered to the eye in the form of an ophthalmic emulsion, ophthalmic liposomes, nano wafers, a nano particle suspension, or an ophthalmic ointment.

In a further embodiment, the pharmaceutical composition, the ophthalmic device, or the method of treating, of any one of the above embodiments and any one or more of the further embodiments herein, wherein the pharmaceutical composition is ophthalmically administered to an eye of the patient via an ophthalmic device.

In a further embodiment, the pharmaceutical composition, the ophthalmic device, or the method of treating, of any one of the above embodiments and any one or more of the further embodiments herein, wherein the pharmaceutical composition is administered 1, 2, 3, 4, or 5, times per day.

In a further embodiment, the pharmaceutical composition, the ophthalmic device, or the method of treating, of any one of the above embodiments and any one or more of the further embodiments herein, wherein the patient is of an age of about 4-18 years.

In a further embodiment, the pharmaceutical composition, the ophthalmic device, or the method of treating, of any one of the above embodiments and any one or more of the further embodiments herein, wherein the patient is of an age of about 16-26 years.

All publications and patent applications mentioned in this specification are herein incorporated by reference in their entirety to the same extent as if each individual publication or patent application was specifically and individually indicated to be incorporated by reference.

It will be understood that the embodiments disclosed and defined in this specification extends to all alternative combinations of two or more of the individual features mentioned or evident from the text or drawings. All of these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An ophthalmic composition, comprising:
i) a muscarinic receptor antagonist; and
ii) an adenosine receptor antagonist.

2. The ophthalmic composition of claim 1, wherein the muscarinic receptor antagonist is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolomine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidylbenzhexol, tolterodine, or a pharmaceutically acceptable salt thereof.

3. The ophthalmic composition of claim 1, wherein the muscarinic receptor antagonist is atropine, or a pharmaceutically acceptable salt thereof.

4. The ophthalmic composition of claim 1, wherein the muscarinic receptor antagonist is present in an amount in the range from between approximately 0.001 wt. % to less than 0.05 wt. %, relative to the ophthalmic composition.

5. The ophthalmic composition of claim 1, wherein the adenosine receptor antagonist is a xanthine derivative, or a pharmaceutically acceptable salt thereof.

6. The ophthalmic composition of claim 1, wherein the adenosine receptor antagonist is caffeine, or a pharmaceutically acceptable salt thereof.

7. The ophthalmic composition of claim 1, wherein the adenosine receptor antagonist is present in an amount in the range of between approximately 0.1-5.0 wt. %, relative to the ophthalmic composition.

8. The ophthalmic composition of claim 1, wherein the muscarinic receptor antagonist is atropine and is present at a concentration in the range of approximately 0.01-0.04%, an adenosine receptor antagonist is caffeine and is present at a concentration in the range of approximately 0.5-3.0%, relative to the ophthalmic composition.

9. The ophthalmic composition of claim 1, wherein:
i) the ophthalmic composition does not increase the photopic pupil size of an eye beyond 2 mm; and/or
ii) the ophthalmic composition does not decrease the amplitude of accommodation of the eye beyond about 6.0D.

10. The ophthalmic composition of claim 1, wherein the ophthalmic composition is a topical ophthalmic composition.

11. The ophthalmic composition of claim 1, wherein the ophthalmic composition is contained within an ophthalmic device.

12. The ophthalmic composition of claim 11, wherein the ophthalmic device is a contact lens, an ocular insert, a corneal onlay, a corneal inlay, a nano wafer, a liposome, a nanoparticle, a punctal plug, or a hydrogel matrix with microfluid reservoir.

13. A method of treating myopia in a patient in need thereof, comprising: administering the ophthalmic device of claim 11.

14. A method of treating myopia in a patient in need thereof, comprising: administering the ophthalmic composition of claim 1.

15. The method of treating of claim 14, wherein the ophthalmic composition is topically administered to the eye in the form of an eye drop formulation, an ocular spray formulation, or an ocular gel formulation.

16. The method of treating of claim 13, wherein the method slows or reduces the progression of myopia in the treated patient, relative to non-treatment.

17. The method of treating of claim 13, wherein the method:
i) increases choroidal thickness of an eye of the treated patient, relative to non-treatment; and/or
ii) reduces axial (or longitudinal) growth of an eye of the treated patient, relative to non-treatment.

18. The method of treating of claim 13, wherein the treated patient suffers from less severe adverse side effects, relative to atropine monotherapy.

19. The method of treating of claim 13, wherein the method does not increase the photopic pupil size of the eye beyond about 2 mm.

20. The method of treating of claim 13, wherein the method does not decrease the amplitude of accommodation of the eye beyond about 6.0D.
